(19) United States
(12) Patent Application Publication
(10) Pub. No.: US 2025/0064799 A1
(43) Pub. Date: Feb. 27, 2025

(54) ORAL FORMULATIONS OF LEVOSIMENDAN FOR TREATING PULMONARY HYPERTENSION WITH HEART FAILURE WITH PRESERVED EJECTION FRACTION

(71) Applicant: Tenax Therapeutics, Inc., Chapel Hill, NC (US)

(72) Inventors: Stuart Rich, Skokie, IL (US); Douglas Randall, Wake Forest, NC (US); Douglas Hay, Ottsville, PA (US)

(73) Assignee: Tenax Therapeutics, Inc., Chapel Hill, NC (US)

(21) Appl. No.: 18/725,602

(22) PCT Filed: Dec. 29, 2022
(86) PCT No.: PCT/US2022/082561

§ 371 Date: Jun. 28, 2024

Related U.S. Application Data
(60) Provisional application No. 63/304,201, filed on Jan. 28, 2022.
(60) Provisional application No. 63/295,760, filed on Dec. 31, 2021.

(51) Int. Cl. A61K 31/50; A61B 5/00; A61K 9/00; A61K 45/06; A61P 9/12

(52) U.S. Cl. CPC A61K 31/50; A61B 5/4848; A61K 9/0053; A61K 45/06; A61P 9/12

(57) ABSTRACT

This invention relates to the treatment of Pulmonary Hypertension with heart failure with preserved ejection fraction (PH-HFpEF). More specifically, embodiments of the invention provide compositions and methods useful for the treatment of PH-HFpEF employing the use of orally administered levosimendan.

This application claims the benefit of U.S. Provisional Application No. 63/304,201, filed Jan. 28, 2022 and U.S. Provisional Application No. 63/295,760, filed Dec. 31, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### FIELD OF THE INVENTION

The invention relates to the treatment of heart failure with preserved ejection fraction, specifically in human subjects who also have pulmonary hypertension (i.e. PH-HFpEF patients), with orally administered levosimendan.

### BACKGROUND OF THE INVENTION

### Levosimendan

Levosimendan is approved in over 60 countries for intravenous use in hospitalized subjects with acutely decompensated heart failure (ADHF). Levosimendan is currently approved for in-hospital use only, and currently approved only for administration in a hospital setting where adequate monitoring facilities and expertise with the use of inotropic agents are available. (Simdax. Finland: Orion Corporation; 2010.)

Levosimendan enhances the calcium sensitivity of contractile proteins by binding to cardiac troponin C in a calcium-dependent manner. Levosimendan increases the contraction force but does not impair ventricular relaxation. In addition, levosimendan opens ATP-sensitive potassium channels in vascular smooth muscle, thus inducing vasodilatation of systemic and coronary arterial resistance vessels and systemic venous capacitance vessels. Levosimendan is also a selective phosphodiesterase III inhibitor in vitro. (Simdax. Finland: Orion Corporation; 2010)

Levosimendan has been studied exclusively in heart failure patients with reduced ejection fraction (HFrEF). In fact, with the single exception of the Hemodynamic Evaluation of Levosimendan in HP-HFpEF (HELP) Study (Borlaug 2020, Burkhoff 2020) that this invention is based upon, all of the many prior multicenter randomized placebo-controlled trials of levosimendan in heart failure patients have specifically excluded heart failure patients with preserved ejection fraction (HFpEF). The complete lack of clinical research evaluating levosimendan in HFpEF and PH-HFpEF patients is consistent with the historical treatment paradigm that levosimendan should be used to treat HFrEF patients. The HELP Study represents a major departure from this traditional mindset and as a result the findings from this novel clinical trial represent significant and surprising discoveries regarding the benefits of levosimendan in PH-HFpEF patients.

In HFrEF patients, the positive inotropic and vasodilatory actions of levosimendan result in an increased contractile force, and a reduction in both preload and afterload, without adversely affecting diastolic function. Hemodynamic studies in healthy volunteers and in patients with stable and unstable heart failure have shown a dose-dependent effect of levosimendan given intravenously as loading dose (3 micrograms/kg to 24 micrograms/kg) and continuous infusion (0.05 to 0.2 micrograms/kg per minute). Compared with placebo, in HFrEF patients levosimendan increased cardiac output, stroke volume, ejection fraction, and heart rate and reduced systolic blood pressure, diastolic blood pressure, pulmonary capillary wedge pressure, right atrial pressure, and peripheral vascular resistance. (Simdax. Finland: Orion Corporation; 2010.)

Levosimendan's activity is mediated through unique mechanisms of action, including: increased cardiac contractility by calcium sensitization of troponin C, vasodilation through opening of potassium channels, and cardioprotective effects via potassium channel opening in mitochondria. (Haikala et al. 1995, Haikala et al. 1995, Pollesello et al. 1994, Sorsa et al. 2004, Yokoshiki et al. 1997, Pataricza et al. 2000, Kaheinen et al. 2001, Erdei et al. 2006, Maytin et al. 2005, Pollesello et al. 2007, du Toit et al. 2008, Louhelainen et al. 2010)

Levosimendan has been shown to be a potent and selective phosphodiesterase-3 (PDE3) inhibitor in vitro. The drug is PDE3 selective with a PDE3/PDE4 inhibition ratio of 10,000. However, both isozymes must be inhibited in cardiomyocytes to exert an effect on the cAMP concentration and inotropic effects. The classical PDE inhibitors (i.e., milrinone, enoximone, and amrinone) inhibit both PDE3 vs. PDE4 is as low as 17-fold), which accounts fully for their inotropic effect. (Yokoshiki et al. 1997, Szilagyi et al. 2004)

Levosimendan improves endothelial function and enhances diastolic coronary flow by opening the adenosine triphosphate-sensitive potassium channels and increasing nitric oxide production. Levosimendan acts through direct binding to troponin-C at high systolic intracellular calcium concentration as well as detachment from it at low diastolic concentration are facilitated. Levosimendan displayed positive lusitropic effects relative to milrinone and nitroglycerin. The lusitropic effect of levosimendan is independent of the degree of the inotropic effect. (Michaels et al. 2005, Grossini et al. 2005, Hasenfuss et al. 1998, De Luca et al. 2006)

### Metabolites OR-1896 and OR-1855

Levosimendan has an active metabolite that extends its effects well beyond the infusion period. Following intravenous or oral dosing, levosimendan is reduced by intestinal bacteria to form OR-1855 (limited activity) that is acetylated to form OR-1896, an active metabolite. While the parent half-life is approximately 1 hour and cleared a few hours after the end of intravenous infusion, OR-1896 has a prolonged half-life of 70-80 hours in heart failure subjects with roughly equal exposures of OR-1855 and OR-1896 maintained through deacetylation/acetylation pathways. The OR-1896 metabolite has been shown to retain similar hemodynamic and pharmacologic properties of levosimendan and maintain roughly equivalents to levosimendan in preclinical models. This activity occurs despite considerably lower plasma concentrations relative to levosimendan, an apparent result of a large percentage of unbound OR-1896 in circulation. Thus, in extended repeated dosing, levosimendan is essentially an active prodrug to an active metabolite moiety, OR-1896. (Louhelainen et al. 2010, Erdei et al. 2006, Szilagyi et al. 2004, Banfor et al. 2008, Louhelainen et al. 2009, Segreti et al. 2008)

OR-1896 is equipotent to levosimendan in its inotropic effects in whole cardiomyocytes and isolated contractile apparatus preparations. However, OR-1896 is profoundly less potent in the inhibition of both PDE3 and PDE4 isozymes. This supports the hypothesis that the main component of the inotropic effect for both levosimendan and OR-1896 is a result of their binding to troponin C and not through PDE inhibition. (Szilagyi et al. 2004)

Clinical observations demonstrate that short-term levosimendan administration is followed by long-term hemodynamic changes that parallel the levels of OR-1896. Patients have been observed with detectable concentrations of both metabolites, OR-1896 and OR-1855, in follow-ups two weeks after treatment. Despite OR-1855's observed inactivity, OR-1896 greatly extends the parent levosimendan's activity and provides the primary active moiety in subjects receiving intermittent intravenous levosimendan therapy. (Banfor et al. 2007, Kivikko et al. 2003, Kivikko et al. 2002)

Based on knowledge of OR-1896 and OR-1855, administration of the metabolites could be used analogously to levosimendan, with adjustments made for the metabolites' own parameters. Both metabolites could be delivered through various routes of administration, including but not limited to, oral, intravenous, and subcutaneous administration. The dose chosen depends on the specific route of administration. In all cases, the target dosing would be intended to achieve a steady-state concentration of OR-1896 of 0.5 to 25.0 ng/ml. The relationship between levosimendan and OR-1896 and OR-1855, along with the interaction between the metabolites, is discussed in Pharmacodynamics and Safety of a New Calcium Sensitizer, Levosimendan, and Its Metabolites during an Extended Infusion in Patients with Severe Heart Failure (Kivikko et al. 2002), the entire contents of which are incorporated by reference.

Types of Heart Failure—HFrEF vs. HFpEF

HFpEF and HFrEF are distinct clinical entities, and as noted above, clinical trials of levosimendan conducted prior to the HELP Study explicitly excluded HFpEF patients. While each type of heart failure accounts for approximately 50% of all heart failure patients, many differences exist between these two forms of heart failure.

A recent review by Shah et al. described some of the distinct features of HFpEF and HFrEF, summarized in the chart below. The review noted that over the past three decades, HFrEF evolved into its own distinct therapeutic entity due to the efficacy of neurohormonal inhibition seen in large outcome clinical trials. However, HFpEF has not undergone a similar evolution due to the consistent failure of large trials testing neurohormonal inhibition either individually or on meta-analysis. (Shah et al. 2016)

Unequal Structural, Functional, and Ultra-structural
LV Characteristics in HFpEF and HFrEF

| | |
|---|---|
| HFpEF | HFrEF |

LV structure/function

| | | |
|---|---|---|
| End-diastolic volume | ↔ | ↑ |
| End systolic volume | ↔ | ↑ |
| Wall thickness | ↑ | ↔ |
| Mass | ↑ | ↑ |
| Mass/volume ratio | ↑ | ↓ |
| Remodeling | Concentric | Eccentric |
| Ejection fraction | ↔ | ↓ |
| Stroke work | ↔ | ↓ |
| End-systolic elastance | ↔ | ↓ |
| End-diastolic stiffness | ↑ | ↓ |

LV ultrastructure

| | | |
|---|---|---|
| Myocyte diameter | ↑ | ↔ |
| Myocyte length | ↔ | ↑ |
| Myocyte remodeling | Concentric | Eccentric |
| Fibrosis | Interstitial/reactive | Focal/replacement |

Pulmonary Hypertension—Heart Failure with Preserved Election Fraction (PH-HFpEF)

Many HFpEF patients have coexisting Pulmonary Hypertension. A sustained elevation in left atrial pressure causes pulmonary venous congestion, which often leads to elevation of pulmonary pressures leading to severe right ventricular failure with a low cardiac output, edema, hypoxemia, and severely limited exercise capacity. Pulmonary hypertension (PH) in subjects with heart failure and preserved ejection fraction (PH-HFpEF) is a common form of pulmonary hypertension and has an estimated US prevalence exceeding 1.5 million. (Oktay et al. 2013, Oudiz et al. 2007, Hoeper et al. 2016)

PH-HFpEF has been classified within Group II of the World Health Organization (WHO) clinical classification of PH, characterized by PH arising from left heart disease. Regardless of the basis of left heart disease, PH initially develops from a passive backward transmission of filling pressures, mainly driven by left ventricular (LV) diastolic function, resulting in a chronic increase in left atrial pressure and a loss of left atrial compliance. These mechanical components of pulmonary venous congestion may trigger pulmonary vasoconstriction, decreased nitric oxide (NO) availability, increased endothelin expression, desensitization to natriuretic peptide-induced vasodilation, and vascular remodeling. Finally, these changes often lead to advanced pulmonary vascular disease, increased right ventricle (RV) afterload, and RV failure. PH-HFpEF is defined hemodynamically by a pulmonary artery pressure (mPAP) ≥25 mmHg, a pulmonary capillary wedge pressure (PCWP) >15 mmHg, and a diastolic pressure gradient [diastolic PAP−PCWP]>7 mmHg. (Galie et al. 2009, McLaughlin et al. 2009, Simonneau et al. 2009, Dixon et al. 2015)

ESC guidelines in the treatment of PH-HFpEF subjects acknowledge that the accepted treatment target is a reduction of pulmonary wedge pressures using diuretics for congestion. However, clinical studies have demonstrated neutral results with identified concerns that Pulmonary Hypertension (PH)-targeted therapies could have detrimental effects due to rapid increases in LV filling pressures, resulting in acute pulmonary edema. Thus, the ESC guidelines specify that there are currently no established strategies to treat pulmonary vascular disease (PVD) and right ventricular disease (RVD) in HFpEF, with a recommendation (class III) not to use approved PAH treatments in PH-HFpEF subjects. With no demonstrated effective therapy, these subjects have a poor outcome (5 yr. survival <50%, frequent hospitalizations). (Shah et al. 2016, Galie et al. 2009, Gorter et al. 2018, Klapholz et al. 2004). A recent comprehensive review of the scientific literature regarding PH-HFpEF stated the following regarding the lack of effective therapies: “[N]o evidence based therapies exist for PH attributable to HFpEF (PH-HFpEF), in part because the pathophysiology is poorly understood” (Brittain et al 2022).

Levosimendan has never previously been studied in the PH-HFpEF population. The complete lack of research regarding levosimendan's potential utility in PH-HFpEF likely stems from the fact that inotropes such as levosimendan, are recommended in most heart failure guidelines to be used exclusively in the treatment of HFrEF and not HFpEF patients. As an example, the 2013 ACCF/AHA guidelines for management of heart failure specifically limits its recommendation for inotrope use to HFrEF patients, stating “Use of parenteral inotropic agents in hospitalized patients without documented severe systolic dysfunction, low blood pressure, or impaired perfusion and evidence of significantly depressed cardiac output, with or without congestion, is potentially harmful.”

### Failed Treatment Attempts

Additionally, none of the currently approved drugs used to treat other forms of pulmonary hypertension have shown to be effective in PH-HFpEF. In fact, all prior trials that have tested other drugs in PH-HFpEF have repeatedly reported neutral to negative results. (ElGuindy et al. 2012).

Numerous review articles have been published attempting to consolidate the repeated failures in attempted treatments of PH-HFpEF. One, in particular, Pulmonary Vascular Disease in the Setting of Heart Failure with Preserved Ejection Fraction, written by Andrea R. Levine et al., consolidated all the background information behind the disease while also applying this background information to previously failed attempts. The comments on failed attempts of various therapeutics is referenced below to help explain the vast difficulty in treating PH-HFpEF.

One targeted pathway in previous clinical trials was the Nitrate-NO-sGC-cGMP pathway. While several small, single-center trials have reported positive results treating PH-HFpEF with PDE5i, a large multicenter study was negative, making it unlikely that PDE5i will ever be an approved therapy for PH-HFpEF. In 2015, Hoendermis et al. found no change in mPAP after 12 weeks of sildenafil administration in 52 patients. The RELAX trial sought to establish whether chronic sildenafil administration changed peak oxygen consumption at 24 weeks in patients with HFpEF. However, long term sildenafil treatment failed to improve six-minute walk time, clinical status, or quality of life in this multicenter trial of 216 patients. The SIOVAC trial was a multicenter placebo control trial of sildenafil in patients with PH-left heart disease (LHD) secondary to valvular heart disease. This study reinforced the risk associated with use of sildenafil in patients with PH-LHD, and it also supported the recommendations against the use of PDE5i in patients with PH-LHD. Due to these negative results seen to date, the efficacy of PDE5i in PH-HFpEF seems highly unlikely. The multicenter INDIE-HFpEF studied the acute cardiopulmonary hemodynamic effects of inorganic nitrite infusion. However, this study was also unsuccessful, as it was unable to demonstrate any improvement in the primary endpoint of peak oxygen consumption during cardiopulmonary exercise or secondary endpoints including activity level, quality of life score, or NT-proBNP in patients treated with inhaled sodium nitrite 3 times a day for 4 weeks. Another study, conducted by Simon et al., further evaluated inhaled nitrites in PH-HFpEF patients with some promising data related to cardiopulmonary hemodynamics; however, improvements in clinical endpoints have not been demonstrated to date. The DILATE trial and SOCRATES-PRESERVED trial both assessed the acute hemodynamic effects of sGC stimulators, riociguat and vericiguat respectively. Although the DILATE trial showed some promising results, the primary outcome was not achieved in mean pulmonary arterial pressure, along with no change in TPG or pulmonary vascular resistance. The SOCRATES-PRESERVED trial ended with similar results, where the primary endpoints also saw no significant changes. (Levine et al. 2019)

Endothelin receptors are another previously targeted molecular target for treatment of PH-HFpEF. MELODY-1 was a small pilot study evaluating macitentan in patients with left heart disease. However, primary outcomes were fluid retention and worsening of the New York Heart Association (NYHA) functional class. Additionally, no change was seen in hemodynamic parameters such as pulmonary vascular resistance, mean pulmonary arterial pressure, or pulmonary artery wedge pressure. The BADDHY trial also attempted using an endothelin receptor antagonist, bosentan, in patients with PH-HFpEF, but no improvements were seen in the six minute walk test or echocardiographic evaluation of pulmonary hypertension. Patients who received bosentan actually had worse clinical outcomes than those who only received the placebo. Neither of these two studies indicated any success with endothelin receptor antagonists in treating PH-HFpEF. (Levine et al. 2019)

The largest clinical trial in HFpEF to date was the PARAGON-HF trial conducted by Novartis. This trial was designed to evaluate the effect of sacubitril/valsartan on HFpEF patients. PARAGON-HF was yet another example of a large clinical trial where the reduction in the primary endpoint was not statistically significant. (Novartis 2019)

According to 2009 ACCF/AHA and 2015 ESC/ERS Guidelines, there is no current clinically approved treatment for PH-HFpEF. Given the numerous amount of failures and adverse effects known in the field as summarized above, no drug can be expected to treat PH-HFPEF, but there is a strong need in the field to find a treatment for this currently untreatable disease. (Levine et al. 2019).

### SUMMARY OF THE INVENTION

The invention relates to the treatment of Pulmonary Hypertension with heart failure with preserved ejection fraction (PH-HFpEF). More specifically, embodiments of the invention provide compositions and methods useful for the treatment of PH-HFpEF, employing the use of orally administered levosimendan, or OR1896, or OR1855. Other objects, features and advantages of the present invention will become clear from the following description and drawings.

### BRIEF DESCRIPTION OF THE DRAWINGS

FIG. 1 shows the changes in NT-proBNP or BNP measurements by each patient in the transition study.

FIG. 2 shows a waterfall plot of the change from baseline to Week 6 in KCCQ Summary Scores-Safety Population.

FIG. 3: Pulmonary Capillary Wedge Pressure (PCWP) of Patient 019-022 of HELP Study. Patient 019-022 showed a decrease in pulmonary capillary wedge pressure (PCWP) after levosimendan administration. These results are based on the HELP Study single 24-hour open label lead-in infusion data.

FIG. 4: 6-Minute Walk Distance of Patient 019-022 of HELP Study. Patient 019-022 showed a significant improvement in their 6-minute walk distance after receiving a combination therapy of levosimendan and empagliflozin.

FIG. 5: 6-Minute Walk Distance Change during single agent therapy (Levosimendan alone) vs. combination therapy of Empagliflozin and Levosimendan. Data from the HELP Study and Patient 019-002 are shown to compare the effects of treatment with levosimendan alone and the combination of empagliflozin and levosimendan on exercise capacity as measured by 6-minute walk distance changes.

FIGS. 6A-6W: Pharmacokinetic Data from the Levosimendan I.V. to Oral Transition Study. FIG. 6A shows consistently higher OR-1896 plasma concentration (ng/ml, y-axis) across patients (x-axis) is achieved with 3 mg daily oral levosimendan administration (Week 6) compared to weekly I.V. levosimendan administration (Week 0). FIG. 6B shows individual patient OR-1896 plasma concentrations (ng/ml, y-axis) at Week 0 (weekly I.V. levosimedan) vs. Week 6 (daily 3 mg oral levosimendan). FIG. 6C shows individual patient OR-1896 plasma concentrations (ng/ml, y-axis) by acetylation status (rapid, intermediate, or slow). FIGS. 6D-6U show patient OR-1896 plasma concentrations (ng/ml, y-axis) for each patient in the I.V. to oral transition study. The respective patient number is indicated above each graph. FIG. 6V: Individual Patient Change in 6-minute walk distance (6MWD) at Week 6 (y-axis) by OR-1896 plasma concentration (Week 6) (x-axis). FIG. 6W: Individual Patient Change in Heart Rate (Week 0 to Week 6) (y-axis) by OR-1896 plasma concentration (Week 6) (x-axis).

FIG. 7: Transition study final dosing.

FIG. 8: Change in heart rate. Mean change in resting heart rate.

FIG. 9: 6-Minute Walk Distance (6MWD).

FIG. 10: Change in BNP. Mean A BNP.

FIG. 11: Change in NT-BNP.

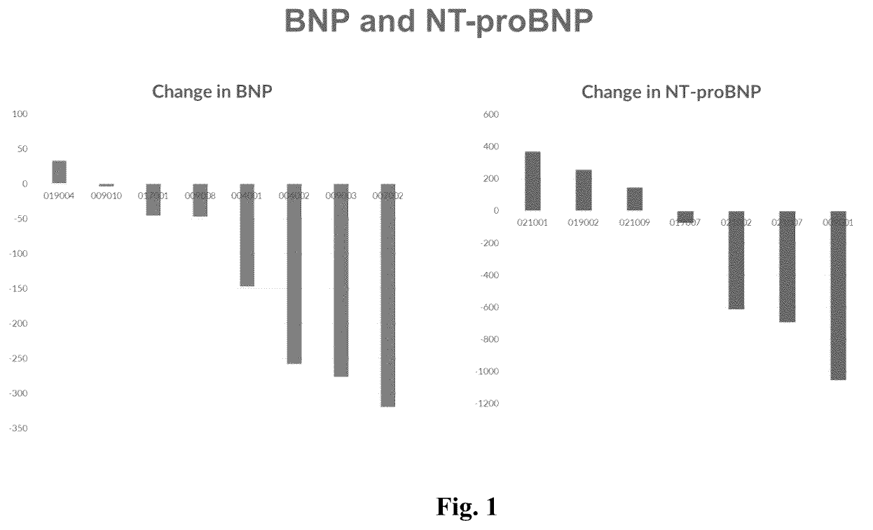

### DETAILED DESCRIPTION OF THE INVENTION

According to some embodiments, the invention provides a method for treating Pulmonary Hypertension Heart Failure with preserved ejection fraction (PH-HFpEF) in a human subject afflicted with PH-HFpEF comprising orally administering to the human subject an amount of levosimendan, its metabolites OR-1896 or OR-1855, or a combination thereof, that is effective to treat the PH-HFpEF in the human subject.

In some embodiments, the treating comprises
a) a reduction in the human subject's pulmonary capillary wedge pressure at rest, preferably by 1 to 30 mmHg;
b) a stabilization of the human subject's pulmonary capillary wedge pressure at rest at 5 to 35 mmHg or 10 to 35 mmHg;
c) a reduction in the human subject's pulmonary capillary wedge pressure during exercise by the human subject, preferably by subject by 1 to 40 mmHg;
d) stabilization of the human subject's pulmonary capillary wedge pressure during exercise by the human subject, preferably at 10 to 50 mmHg;
e) the treating does not comprise a significant change in pulmonary capillary wedge pressure during exercise by the human subject;
f) a reduction in the human subject's pulmonary capillary wedge pressure when the human subject's legs are elevated, preferably the reduction is 1 to 30 mmHg;
g) stabilization of the human subject's pulmonary capillary wedge pressure when the human subject's legs are elevated, preferably the stabilization is at 10 to 50 mmHg;
h) a reduction in the human subject's right atrial pressure at rest, preferably by 1 to 30 mmHg;
i) stabilization of the human subject's right atrial pressure at rest at 1 to 30 mmHg or at 5 to 30 mmHg;
j) a reduction in the human subject's right atrial pressure during exercise by the human subject, preferably by 1 to 30 mmHg;
k) stabilization of the human subject's right atrial pressure during exercise by the human subject at 5 to 40 mmHg;
l) a reduction in the human subject's right atrial pressure when the human subject's legs are elevated;
m) a reduction in the human subject's mean pulmonary artery pressure at rest, preferably by 1 to 30 mmHg;
n) stabilization of the human subject's mean pulmonary artery pressure at rest at 15 to 65 mmHg;
o) a reduction in the human subject's mean pulmonary artery pressure during exercise by the human subject, preferably by 1 to 30 mmHg;
p) stabilization of the human subject's mean pulmonary artery pressure during exercise by the human subject at 25 to 85 mmHg or 25 to 80 mmHg;
q) a reduction in the human subject's mean pulmonary artery pressure when the human subject's legs are elevated;
r) an increase in the human subject's cardiac output at rest, preferably by 0.01 to 3 liters/min;
s) stabilization of the human subject's cardiac output at rest at 2 to 10 liters/min;
t) an increase in the human subject's cardiac output during exercise by the human subject;
u) an increase in the human subject's cardiac output during exercise by the human subject by 0.01 to 5 liters/min or by 0.01 to 4 liters/min or by at 3.0 to 15.0 liters/min;
v) does not comprise a significant increase in the human subject's heart rate or does not comprise an increase in the human subject's heart rate of more than 10 beats/min;
w) an improvement in the human subject's quality of life;
x) an improvement in the human subject's six (6) minute walk distance, preferably of 5 to 150 meters;
y) an improvement in the physician's assessment of the human subject's functional class;
z) a reduction in the incidence of hospitalization for heart failure;
aa) a reduction in all-cause mortality; or
bb) an improvement in right heart failure and/or right ventricular dysfunction, preferably as evidenced by a reduction in right atrial pressure at rest and during 25 watts of exercise.

In some embodiments, the improvement in the human subject's quality of life is measured by a patient reported outcome assessment tool.

In some embodiments, the treating comprises an improvement in the human subject's quality of life according to a change in the human subject's patient reported outcome assessment tool score of at least 1, more preferably at least 2.

In some embodiments, the human subject is a responder to levosimendan therapy.

In some embodiments,
a) a responder to levosimendan therapy is a human subject whose pulmonary capillary wedge pressure decreases by at least 4 mmHG during bicycle exercise at 25 watts following the initial administration;
b) a responder to levosimendan therapy is a human subject whose cardiac index decreases by no more than 10% between the baseline measurements and repeated measurements following the initial administration;
c) the human subject is a responder to levosimendan therapy if the human subject has cardiac reserve;
d) the human subject is a responder to levosimendan therapy if the human subject's stroke volume increases during exercise by the human subject;
e) the human subject is a responder to levosimendan therapy if the human subject's stroke volume increases during exercise by the human subject when determined with a catheter in the human subject's heart measuring the blood moving out of the left ventricle with every beat;
f) the human subject is a responder to levosimendan therapy if the human subject's stroke volume increases during exercise by the human subject when estimated with an electrocardiogram and/or echocardiogram;
g) the human subject is a responder to levosimendan therapy if the human subject's stroke volume increases during exercise by the human subject when determined with a dobutamine stress test;
h) the human subject is a responder to levosimendan therapy if the human subject's stroke volume increases during exercise by the human subject by at least 0.005 liters;
i) the human subject is a responder to levosimendan therapy if the human subject's stroke volume increases during exercise by the human subject by 1 to 50 mL when determined with a catheter in the human subject's heart measuring the blood moving out of the left ventricle with every beat;
j) the human subject is a responder to levosimendan therapy if the human subject's stroke volume increases during exercise by the human subject by 1 to 50 mL when estimated with an echocardiogram, right heart catheterization, or other means; or
k) the human subject is a responder to levosimendan therapy if the human subject's stroke volume increases during exercise by the human subject by 1 to 50 mL when determined with a dobutamine stress test.

In some embodiments, wherein the human subject afflicted with PH-HFpEF
a) has a left ventricular ejection fraction of at least 40%;
b) has a baseline pulmonary arterial pressure of at least 35;
c) has a baseline pulmonary capillary wedge pressure of at least 20;
d) is classified as classification IIb or classification III by the physician's assessment of New York Heart Association Classification;
e) has the ability to walk at least 50 meters in a six-minute walk test, does not have the ability to walk more than 550 meters in a six-minute walk test, or has the ability to walk at least 50 meters, but not more than 550 meters, in a six-minute walk test;
f) is not afflicted with heart failure with reduced ejection fraction;
g) is not afflicted with heart failure with preserved ejection fraction without pulmonary hypertension;
h) has a primary diagnosis of Group 2 PH-HFpEF;
i) is not afflicted with coronary artery disease;
j) has not had previous percutaneous coronary intervention;
k) has not had previous percutaneous coronary intervention, unless the human subject has had a negative stress test within the last year;
l) has not had previous cardiac surgery;
m) has not had previous cardiac surgery, unless the human subject has had a negative stress test within the last year;
n) is not afflicted with congenital heart disease;
o) is not afflicted with a clinically significant lung disease;
p) does not have a planned heart or lung surgery;
q) does not have a cardiac index greater than 4.0 L/min/m2;
r) does not concomitantly receive pulmonary vasodilator therapy;
s) has not received pulmonary vasodilator therapy within the last 14 days;
t) does not receive dialysis treatment;
u) does not have a Glomerular Filtration Rate less than 30 mL/min/1.73 m2;
v) does not have liver dysfunction with Child Pugh Class B or C;
w) does not have evidence of systemic infection;
x) does not weigh more than 150 kg;
y) can manage their symptomatic systolic blood pressure to ensure it is greater than 100 mmHg;
z) does not have a heart rate greater than or equal to 100 beats per minute with the drug;
aa) does not have a heart rate greater than or equal to 100 beats per minute with the drug that is symptomatic and persistent for at least 10 minutes;
bb) does not have hemoglobin less than 80 g/L;
cc) does not have serum potassium less than 3.0 mmol/L at baseline;
dd) does not have serum potassium greater than 5.5 mmol/L at baseline;
ee) does not have serum potassium less than 3.0 mmol or greater than 5.5 mmol/L at baseline;
ff) does not have severely compromised immune function;
gg) is not pregnant, is not suspected to be pregnant, or is not breast-feeding; or
hh) is a patient with Biventricular Failure.

In some embodiments, the administering takes place once daily, twice daily, three times daily, four times daily, intermittently, weekly, or chronically.

In some embodiments, the oral administration comprises an immediate release formulation, modified release formulation, or an extended-release formulation.

In some embodiments, the amount of levosimendan its metabolites OR-1896 or OR-1855, or a combination thereof, is administered in combination with a cardiovascular drug.

In some embodiments, the amount of levosimendan its metabolites OR-1896 or OR-1855, or a combination thereof, and the amount of the cardiovascular drug when taken together is effective to reduce the symptoms of PH-HFpEF.

In some embodiments, the cardiovascular drug is a drug used to treat pulmonary arterial hypertension (PAH), World Health Organization (WHO) Groups 1-5 pulmonary hypertension patients, coronary artery disease (CAD), or heart failure with reduced ejection fraction (HFrEF).

In some embodiments, the cardiovascular drug is a PDE inhibitor, a phosphodiesterase-5 (PDE5) inhibitor, an endothelin receptor antagonist (ERA), a prostanoid, a soluble guanylate cyclase stimulator, a nitrate, a nitrite, an NO donor, a calcium channel blocker (CCB), a fatty acid oxidation inhibitor, a beta-blocker (BB), an angiotensin-converting enzyme (ACE) inhibitor, a neprilysin inhibitor, a neprilysin and angiotensin receptor blocker (ANRI), an angiotensin II receptor blocker (ARB), a diuretic, an aldosterone antagonist, digoxin, ivabradine, hydralazine, seralaxin, a natriuretic peptide, an atrial natriuretic peptide (ANP), a natriuretic peptide, a K-ATP channel activator, a NEP inhibitor, or a prostacyclin.

In some embodiments, the cardiovascular drug is a pulmonary vasodilator drug.

In some embodiments, the pulmonary vasodilator is a phosphodiesterase-5 (PDE5) inhibitor, an endothelin receptor antagonist (ERA), or a prostacyclin.

In some embodiments, the amount of levosimendan, its metabolites OR-1896 or OR-1855, or a combination thereof, administered in combination with the pulmonary vasodilator drug is administered to a human subject afflicted with pre and post capillary pulmonary hypertension and heart failure with preserved ejection fraction (Cpc-PH-HFpEF).

In some embodiments, no atrial arrhythmias or ventricular arrhythmias is observed when comparing baseline electrocardiographic monitoring with 72-hour monitoring after 5 weeks oftreatment.

In some embodiments, treating presents no more statistically significant adverse events than the matching placebo.

In some embodiments, the subject is orally administered a capsule comprising up to 0.1 mg, 0.25 mg, 0.5 mg, 0.75 mg, 1 mg, 2 mg, 3 mg, or 4 mg, more preferably 1-3 mg, of levosimendan, its metabolites OR-1896 or OR-1855, or a combination thereof.

In some embodiments, the subject is administered a capsule once a day, twice a day, three times a day, or four times a day for a time period of 1-60 days, preferably 14 days.

In some embodiments, the subject increases the number of capsules taken per day after every time period if the treatment is tolerated by the subject.

In some embodiments, the subject is orally administered between 0.1-10 mg of levosimendan, its metabolites OR-1896 or OR-1855, or a combination thereof, per day, preferably between 1-4 mg of levosimendan, its metabolites OR-1896 or OR-1855, or a combination thereof per day.

In some embodiments, the subject received a final intravenous injection of levosimendan, its metabolites OR-1896 or OR-1855, or a combination thereof at least one day, more preferably at least one week, before beginning oral administration of levosimendan, its metabolites OR-1896 or OR-1855, or a combination thereof.

In some embodiments, the human subject is administered an effective amount of a combination therapy comprising
a) an amount of levosimendan, its metabolites OR-1896 or OR-1855, or a combination thereof; and
b) an amount of a sodium-glucose cotransporter-2 (SGLT-2) inhibitor.

In some embodiments, comprising periodically administering to the subject an amount of the levosimendan, its metabolites OR-1896 or OR-1855, or a combination thereof, and an amount of the SGLT-2 inhibitor, wherein the amounts when taken together are effective to treat the subject.

In some embodiments, treating the subject with the combination therapy is more effective to treat the subject than when either the amount of levosimendan or the amount of the SGLT-2 inhibitor is administered alone.

In some embodiments, the amounts of levosimendan and the SGLT-2 inhibitor when taken together are effective to achieve a greater than additive therapeutic result in treating the subject.

In some embodiments, the subject was receiving a therapy including levosimendan, its metabolites OR-1896 or OR-1855, or a combination thereof, prior to initiating a SGLT-2 inhibitor therapy.

In some embodiments, the subject was receiving a SGLT-2 inhibitor therapy prior to initiating a therapy including levosimendan, its metabolites OR-1896 or OR-1855, or a combination thereof.

In some embodiments, the amount of SGLT-2 inhibitor is administered first, followed by administration of the amount of levosimendan, its metabolites OR-1896 or OR-1855, or a combination thereof.

In some embodiments, the amount of levosimendan, its metabolites OR-1896 or OR-1855, or a combination thereof, is administered first, followed by administration of a SGLT-2 inhibitor.

In some embodiments, the levosimendan, its metabolites OR-1896 or OR-1855, or a combination thereof, and the SGLT-2 inhibitor are administered sequentially.

In some embodiments, the levosimendan, its metabolites OR-1896 or OR-1855, or a combination thereof, and the SLGT-2 inhibitor are administered simultaneously.

In some embodiments, the levosimendan, its metabolites OR-1896 or OR-1855, or a combination thereof, and the SLGT-2 inhibitor are administered periodically, chronically, weekly, or intermittently.

In some embodiments, the SGLT-2 inhibitor is administered orally.

In some embodiments, the SGLT-2 inhibitor is selected from the group consisting of empagliflozin, canagliflozin, ertugliflozin, or dapagliflozin.

In some embodiments, the subject is administered between 10-25 mg empagliflozin per day.

In some embodiments, the subject is administered between 5-10 mg dapagliflozin per day.

In some embodiments, the subject is administered between 100-300 mg canagliflozin per day.

In some embodiments, the subject is administered between 5-15 mg ertugliflozin per day.

In some embodiments, the subject is administered between 0.1-10 mg of levosimendan, its metabolites OR-1896 or OR-1855, or a combination thereof, per day, preferably between 1-4 mg of levosimendan, its metabolites OR-1896 or OR-1855, or a combination thereof per day.

In some embodiments, the combination therapy is administered as a fixed dose combination.

In some embodiments, the treating with the combination therapy comprises providing
a) an improvement in the human subject's quality of life;
b) an improvement in the human subject's exercise capacity;
c) an improvement in a physician's assessment of the human subject's functional class;
d) a reduction in the incidence of hospitalization for heart failure; and/or
e) a reduction in cardiovascular death.

In some embodiments, the treating with the combination therapy comprises providing an improvement in the human subject's exercise capacity.

In some embodiments, the improvement in the subject's exercise capacity is an increase of at least 10, 20, 30, 40, 50, 60, 70, 80, or 100 meters in a 6-minute walk distance compared to a baseline 6-minute walk distance before the combination therapy treatment.

In some embodiments, the improvement in the subject's exercise capacity is an increase of at least 10%, 20%, 30%, 40%, or 50% relative to a baseline 6-minute walk distance before the combination therapy treatment.

In some embodiments, the improvement in the subject's exercise capacity is within one, two, three, four, five, six, seven, eight, nine, ten, twenty, thirty, forty, or fifty weeks of the administration of the combination therapy.

In some embodiments, the treating comprises providing an improvement in the human subject's hemodynamic measurements at rest and exercise.

In some embodiments, the subject is transitioned to oral administration of levosimendan from intravenous administration of levosimendan, and the OR-1896 plasma concentration of the subject remains the same or increases after the transition to oral administration of levosimendan.

According to some embodiments, the invention provides a pharmaceutical composition comprising levosimendan, its metabolites OR-1896 or OR-1855, or a combination thereof, a SGLT-2 inhibitor, and a pharmaceutically acceptable carrier.

In some embodiments, the SGLT-2 inhibitor is selected from the group consisting of empagliflozin, canagliflozin, ertugliflozin, and dapagliflozin.

According to some embodiments, the invention provides a use of a SGLT-2 inhibitor in combination or as an add-on with a therapy that includes levosimendan, its metabolites OR-1896 or OR-1855, or a combination thereof, to treat a subject afflicted with PH-HFpEF, wherein the SGLT-2 inhibitor and the levosimendan, its metabolites OR-1896 or OR-1855, or a combination thereof are administered simultaneously, contemporaneously or concomitantly.

In some embodiments, the SGLT-2 inhibitor is selected from the group consisting of empagliflozin, canagliflozin, ertugliflozin, and dapagliflozin.

According to some embodiments, the invention provides a use of a SGLT-2 inhibitor in the manufacturing of a medicament for use in combination with or as an add-on to a therapy that includes levosimendan, its metabolites OR-1896 or OR-1855, or a combination thereof, to treat a subject afflicted with PH-HFpEF, wherein the SGLT-2 inhibitor and the levosimendan, its metabolites OR-1896 or OR-1855, or a combination thereof are administered simultaneously, contemporaneously or concomitantly.

In some embodiments, the SGLT-2 inhibitor is selected from the group consisting of empagliflozin, canagliflozin, ertugliflozin, and dapagliflozin.

According to some embodiments, the invention provides a pharmaceutical composition comprising an amount of a SGLT-2 inhibitor and an amount of levosimendan, its metabolites OR-1896 or OR-1855, or a combination thereof, for use in treating a subject afflicted with PH-HFpEF, wherein the SGLT-2 inhibitor and the levosimendan, its metabolites OR-1896 or OR-1855, or a combination thereof, are administered simultaneously, contemporaneously or concomitantly.

In some embodiments, the SGLT-2 inhibitor is selected from the group consisting of empagliflozin, canagliflozin, ertugliflozin, and dapagliflozin.

According to some embodiments, the invention provides A package comprising
a) a first pharmaceutical composition comprising an amount of levosimendan and a pharmaceutically acceptable carrier;
b) a second pharmaceutical composition comprising an amount of an SGLT-2 inhibitor and a pharmaceutically acceptable cartier; and
c) instructions for use of the first and second pharmaceutical compositions together to treat a subject afflicted with PH-HFpEF.

In some embodiments, the SGLT-2 inhibitor is selected from the group consisting of empagliflozin, canagliflozin, ertugliflozin, and dapagliflozin.

According to some embodiments, the invention provides a pharmaceutical composition in unit dosage form, useful in treating a subject afflicted with PH-HFpEF, which comprises:
a) an amount of levosimendan; and
b) an amount of an SGLT-2 inhibitor,
   wherein the respective amounts of said levosimendan and said SGLT-2 inhibitor in said composition are effective, upon concomitant administration to said subject of one or more said unit dosage forms of said composition, to achieve a greater than additive therapeutic result in treating the subject.

In some embodiments, the SGLT-2 inhibitor is selected from the group consisting of empagliflozin, canagliflozin, ertugliflozin, and dapagliflozin.

According to some embodiments, the invention provides a therapeutic package for dispensing to, or for use in dispensing to, a subject afflicted with PH-HFpEF, which comprises:
one or more unit doses, each such unit dose consisting essentially of:
   i) an amount of levosimendan; and
   ii) an amount of an SGLT-2 inhibitor,
   wherein the respective amounts of said levosimendan and said SGLT-2 inhibitor in said unit dose are effective, upon concomitant administration to said subject, to achieve a greater than additive therapeutic result in treating the subject, and a finished pharmaceutical container therefor, said container containing said unit dose or unit doses, said container further containing or comprising labeling directing the use of said package in the treatment of said subject.

In some embodiments, the SGLT-2 inhibitor is selected from the group consisting of empagliflozin, canagliflozin, ertugliflozin, and dapagliflozin.

According to some embodiments, the invention provides use of an amount of an oral formulation of levosimendan, its metabolites OR-1896 or OR-1855, or a combination thereof, for preparing medicament for administering to a human subject afflicted with Pulmonary Hypertension Heart Failure with preserved ejection fraction (PH-HFpEF to effectively treat PH-HFpEF in the human subject.

According to some embodiments, the invention provides a medicament comprising an amount of an oral formulation of levosimendan, its metabolites OR-1896 or OR-1855, or a combination thereof, for use in effectively treating Pulmonary Hypertension Heart Failure with preserved ejection fraction (PH-HFpEF) in a human subject.

According to some embodiments, the invention provides use of an amount of an oral formulation of levosimendan, its metabolites OR-1896 or OR-1855, or a combination thereof, in combination with a cardiovascular drug to effectively treat Pulmonary Hypertension Heart Failure with preserved ejection fraction (PH-HFpEF) in a human subject.

According to some embodiments, the invention provides Use of an amount of an oral formulation of levosimendan, its metabolites OR-1896 or OR-1855, or a combination thereof, for preparing a medicament in combination with a cardiovascular drug for administering to a human subject afflicted with Pulmonary Hypertension Heart Failure with preserved ejection fraction (PH-HFpEF) to effectively treat PH-HFpEF in the human subject.

According to some embodiments, the invention provides a medicament comprising an amount of an oral formulation of levosimendan, its metabolites OR-1896 or OR-1855, or a combination thereof, for use in combination with a cardiovascular drug to effectively treat Pulmonary Hypertension Heart Failure with preserved ejection fraction (PH-HFpEF) in a human subject.

Throughout this application, where a parameter range is provided, all integers within that range, and tenths and hundredths thereof as appropriate, shall be considered to also be provided and disclosed in this application as being contemplated by the invention. For example, “0.2-5 mg/kg/day” is to be considered as a disclosure of 0.2 mg/kg/day, 0.3 mg/kg/day, 0.4 mg/kg/day, 0.5 mg/kg/day, 0.6 mg/kg/day etc. up to 5.0 mg/kg/day.

According to some embodiments, the compound to be administered (e.g. levosimendan) is in the form of a composition (referred to as the composition of the invention) comprising a therapeutically effective amount of at least one of said compound. As used herein, the term “effective amount” means an amount of compound that is capable of reducing and/or attenuating a disorder or symptom as described herein. The specific dose of a compound administered according to this invention will, of course, be determined by the particular circumstances surrounding the case including, for example, the compound administered, the route of administration, the physiological state of the subject, and the severity of the condition being treated.

Any suitable route may be used to orally administer the medicament or levosimendan of the invention to a subject.

According to some embodiments, suitable administration routes may be systemic routes. According to some embodiments, administering is administering systemically. According to some embodiments, the composition is formulated for systemic administration.

According to another embodiment, administration systemically is through an enteral route. According to another embodiment, administration through an enteral route is oral administration. According to some embodiments, the composition is formulated for oral administration.

In an embodiment, the administration is delivered via oral dosing. The dosing can be an immediate release, modified release, or extended release formulation.

In an embodiment, the amount of levosimendan is effective to treat PH-HFpEF in a human subject.

In an embodiment no clinically meaningful arrythmias, atrial or ventricular, are observed when comparing baseline electrocardiographic monitoring with 72-hour monitoring after 5 weeks of treatment.

In an embodiment the weekly 24-hour dosing of levosimendan results in steady state blood levels of OR1896 in the range of 0.20 ng/mL to 25.00 ng/mL.

### Definitions/Abbreviations

As used herein, the term “levosimendan” means levosimendan base or a pharmaceutically acceptable salt thereof. The active compounds for use according to the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the L-tartrate, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

PH is the abbreviation for Pulmonary Hypertension. PH encompasses a heterogeneous group of disorders with the common feature of elevated pulmonary vascular resistance. (Oldroyd et al. 2019)

HFpEF is the abbreviation for heart failure with preserved ejection fraction. HFpEF is when a patient is afflicted with heart failure while their ejection fraction remains ≥40%. (Kelly et al. 2015)

PH-HFpEF is the abbreviation for Pulmonary Hypertension with heart failure with preserved ejection fraction. PH-HFpEF is defined by a high pulmonary artery pressure, high left ventricular end-diastolic pressure and a normal ejection fraction. (Lai et al. 2019)

PCWP is the abbreviation for pulmonary capillary wedge pressure. PCWP is the pressure measured by wedging a pulmonary catheter with an inflated balloon into a small pulmonary arterial branch. PCWP estimates left atrial pressure. (Peacock et al. 2004)

RAP is the abbreviation for right atrial pressure. RAP is the blood pressure in the right atrium of the heart. RAP reflects the amount of blood returning to the heart and the ability of the heart to pump the blood into the arterial system.

mPAP is the abbreviation for mean pulmonary artery pressure. mPAP is generated by the right ventricle ejecting blood into the pulmonary circulation, which acts as a resistance to the output from the right ventricle.

CVP is an abbreviation for central venous pressure. CVP is the blood pressure in the vena cava that empties into the right atrium. CVP reflects the amount of blood returning to the heart that can be pumped into the arteries of the lung.

PVR is the abbreviation for pulmonary vascular resistance. PVR refers to the resistance in the arteries that supply blood to the lungs. (Schnur 2017)

CO is the abbreviation for cardiac output. CO is the volume of blood being pumped by the heart per unit time. (Vincent 2008)

CI is the abbreviation for cardiac index. CI is a hemodynamic parameter that relates the cardiac output from the left ventricle in one minute to the body surface area. This measurement relates heart performance to the size of the individual. (Shea 2019)

HR is the abbreviation for heart rate. HR is the speed of the heartbeat measured by the number of contractions of the heart per minute. (Heart.org 2015)

PR is the abbreviation for pulse rate. PR is the measurement of the heart rate. (Heart.org 2015)

BP is the abbreviation for blood pressure. BP is the pressure of circulating blood within the major arterial system of the body. (Brezinski 1990)

6MWT is the abbreviation for six-minute walk test. 6MWT is a performance-based test used to measure functional exercise capacity. The 6MWT measures the distance an individual is able to walk over a total of 6 minutes at a constant and normal pace. (Vandoni et al. 2018)

Likert scale is a psychometric scale commonly involved in research that employs questionnaires. In the below-mentioned clinical trial, a six-question, five-point Likert Scale is provided to patients to assess their quality of life. (HELP clinical trial protocol)

ECG is the abbreviation for echocardiogram. An ECG is a record of a person's heartbeat produced by echocardiography. An ECG is a test that uses high frequency sound waves (ultrasound) to make pictures of your heart. (heart.org 2015)

Dobutamine stress test is a form of ECG where stress is induced on the heart by administering dobutamine into a vein to assess the heart's function and structures. This test mimics the effects of exercise on the heart. (Hawthorne et al. 2012)

New York Heart Association Functional Classification provides a simple way of classifying the extent of heart failure. A patient in Class I has no limitation of physical activity. A patient in Class II has slight limitation of physical activity. A patient in Class III has marked limitation of physical activity. A patient in Class IV is unable to carry on any physical activity without discomfort. In addition to these class numbers based of patient symptoms, every patient is assigned a class letter based on an objective assessment. A patient in Class A has no objective evidence of cardiovascular disease. A patient in Class B has objective evidence of minimal cardiovascular disease. A patient in Class C has objective evidence of moderately severe cardiovascular disease. A patient in Class D has objective evidence of severe cardiovascular disease. (Yancy et al. 2013)

Self-administration is administration of the formulation administered by the human subject afflicted with the disease. (HELP clinical trial protocol)

Outpatient setting is a setting where the patients do not require admittance for overnight care. (World Health Organization 2009)

Trained professional indicates a doctor, nurse, home healthcare nurse, or other person with training and/or experience and/or a license in the medical profession.

TEAEs is the abbreviation for Treatment Emergent Adverse Events. The TEAEs of special interest are hypotension, atrial fibrillation, other significant arrhythmia, resuscitated death stroke. Other TEAEs include, but are not limited to, headache, increased heart rate, fatigue, cardiac failure acute, dyspnea, vascular access site pain, muscle spasm, and hypokalemia.

SAEs is the abbreviation for Serious Adverse Events. SAEs include, but are not limited to, infections and infestations, device related infection; infections and infestations, bacteremia; cardiac disorders, cardiac failure acute; and cardiac disorders, cardiac failure acute.

As used herein, the term “acute administration” means administration of a drug, e.g. levosimendan, in a brief period of time, for example, delivery of a single dose of a drug, delivery of doses of a drug in rapid succession, or delivery of a drug on short time scale, preferably less than 48 hours. Acute administration of a drug is generally intended for the drug to have a beneficial effect on a condition in the short-term. For example, acute administration of levosimendan may be performed such that the amount of levosimendan administered is intended for the levosimendan drug to directly improve a condition, e.g. PH-HFpEF, prior to any significant activity of a levosimendan metabolite.

As used herein, the term “chronic administration” means an extended and repeated administration of a drug, e.g. levosimendan. For example, delivery of multiple or repeated doses of a drug over the course of a long-time scale, preferably at least a week. Chronic administration of a drug is generally intended for the drug or its metabolites to have a continued beneficial effect on a condition or to prevent or slow deterioration of a disease-state over time. Chronic administration is often delivered to a subject by the subject themselves (i.e. self-administration), for example, by oral or subcutaneous administration.

### Oral Administration

The present invention also relates to oral administration of levosimendan in an oral formulation to achieve the effects disclosed herein. There are numerous practical advantages with oral administration such as: being easily administered and titrated, facilitating patient control, and reducing nursing burden. See, for example, oral formulations of levosimendan provided in PCT International Application Publication No. WO 2021/126884, the entire contents of which are incorporated by reference.

According to some embodiments, oral administration is in the form of hard or soft gelatin capsules, pills, capsules, tablets, including coated tablets, dragees, elixirs, suspensions, liquids, gels, slurries or syrups and controlled release forms thereof. Thus, the invention provides a method of administering levosimendan in the form of a tablet, a capsule, or in a liquid.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include without limitation capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating, agents. In the case of capsules, tablets and pills, the dosage forms may also comprise buffering agents. Tablets and pills can additionally be prepared with enteric coatings. The term “enteric coating”, as used herein, refers to a coating which controls the location of composition absorption within the digestive system. Non-limiting examples for materials used for enteric coating are fatty acids, waxes, plant fibers or plastics. Liquid dosage forms for oral administration may further contain adjuvants, such as wetting agents, emulsifying and suspending agents, and sweetening, flavoring and perfuming agents.

In an embodiment, the administration is delivered via oral dosing, and the oral dosing can be an immediate release, modified release, or extended release formulation.

In some embodiments, a pharmaceutical composition of levosimendan for treatment in subjects in need thereof, for example, for treatment of heart failure with preserved ejection fraction, specifically in human subjects who also have pulmonary hypertension (PH-HFpEF patients), by oral administration is in a formulation comprising an effective amount of levosimendan, its metabolites OR-1896 or OR-1855, or a combination thereof and one or more additional pharmaceutically acceptable additives.

In an embodiment, the oral formulation comprises levosimendan, its metabolites OR-1896 or OR-1855, or a combination thereof in the amount of 0.1 mg, 0.25 mg, 0.5 mg, 0.75 mg, 1 mg, 2 mg, 3 mg, or 4 mg.

In an embodiment, the oral formulation comprises microcrystalline cellulose.

In an embodiment, the oral formulation comprises alginic acid.

In an embodiment, the oral formulation comprises steric acid.

In an embodiment, the oral formulation is in a capsule form.

In an embodiment, the oral formulation is the capsule form is a HPMC capsule.

In an embodiment the oral formulation comprise in a capsule form and the oral formulation comprises 1 mg levosimendan, 96.4 mg microcrystalline cellulose, 30.0 mg alginic acid, and 5.3 mg stearic acid.

In an embodiment, the oral dosage form comprises levosimendan in the amount of 0.1 mg, 0.25 mg, 0.5 mg, 0.75 mg, 1 mg, 2 mg, 3 mg, or 4 mg, more preferably in the amount of 1-3 mg.

In an embodiment, a subject is orally administered a capsule comprising levosimendan in the amount of 1 mg once per day. The oral dosing may be titrated according to, for example, the effectiveness of the treatment, tolerability, changes in heart rate, and body weight of the subject. The titration of levosimendan may be in 1 mg increments and range from 1-10 mg per day, more preferably between 1-4 mg per day.

The titration of levosimendan administration may occur over the course of days, weeks, or months. The effect of duration at a particular dosage amount on tolerability should also be considered when titrating, e.g. the tolerability of a subject to the levosimendan oral treatment may increase with an increase in duration at a particular dosage amount.

For example, a subject may begin an oral levosimendan treatment course at 1 mg/day (i.e. ingesting one capsule comprising 1 mg levosimendan per day). The subject may maintain a levosimendan dosage of 1 mg/day for two weeks. After two weeks, if the levosimendan dosage is well-tolerated and heart rate is increased <15 BPM, the subject can titrate up to a dosage of 2 mg/day (i.e. ingesting two capsules, each comprising 1 mg levosimendan, per day). The subject may continue to titrate up in increments of 1 mg levosimendan in this manner until an optimal oral dosage is achieved, for example, up to 10 mg of levosimendan per day.

A subject receiving levosimendan by other administration routes, for example, intravenous injection, may be transitioned to an oral dosing scheme. For example, a subject receiving levosimendan by intravenous injection may begin an oral dosing after receiving a final 24-hour infusion of levosimendan. The oral dosing of levosimendan may begin within days or weeks, for example, one week, of the final 24-hour infusion. The oral dosage may begin at 1 mg/day, followed by titration as indicated above.

### Combination Therapy

The administration of two drugs to treat a given condition, such as PH-HFpEF, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 2006). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 2006). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 2006)

As used herein, “combination” means an assemblage of reagents for use in therapy either by simultaneous, contemporaneous, or fixed-dose combination delivery. Simultaneous delivery refers to delivery of an admixture (whether a true mixture, a suspension, an emulsion or other physical combination) ofthe drugs. In this case, the combination may be the admixture or separate containers of the levosimendan and a second agent that are combinedjust prior to delivery. Contemporaneous delivery refers to the separate delivery of the levosimendan and second agent at the same time, or at times sufficiently close together that an additive or preferably synergistic activity relative to the activity of either the levosimendan or the cardiovascular drug alone is observed. Fixed-dose combination delivery refers to the delivery of two or more drugs contained in a single dosage form for oral administration, such as a capsule or tablet.

As used herein, “second agent” for use in combination therapy includes any one of the following: Phosphodiesterase-5 (PDE5) inhibitor, an endothelin receptor antagonist (ERA) (e.g., Bosentan, Ambrisentan), a Prostanoid (e.g., Trepostinil, Selexipag, Ralinepag), a Soluble Guanylate Cyclase stimulator (e.g., Riociguat), a nitrate or nitrite, a calcium channel blocker (CCB), fatty acid oxidation inhibitors (e.g., Ranolazine, Trimetazidine), a beta-blocker (BB), an Angiotensin-converting enzyme (ACE) inhibitor, a neprilysin inhibitor (e.g., Sacubitril, Sampatrilat, Gemopatrilat, Fasidotril, Omapatrilat, Candoxatril), a neprilysin and angiotensin receptor blocker (ANRI) (e.g., Entresto), an Angiotensin II receptor blocker (ARB), a diuretic, an Aldosterone antagonist, Digoxin, Ivabradine, Hydralazine, Seralaxin, a natriuretic peptide, an atrial natriueretic peptide (ANP), or Nesiritide.

The recommended dose and schedule for Entresto is 24/26 mg twice daily (24 mg of sacubitril and 26 mg of valsartan). The dose is doubled every two to four weeks, as tolerated by the patient. The composition recited hereinabove is described in U.S. Pat. Nos. 7,468,390; 8,101,659; 8,404,744; 8,796,331; 8,877,938; and 9,388,134, the entire contents of which are incorporated by reference.

The recommended dose and schedule for Sacubitril is 24 mg twice daily. The dose is doubled every two to four weeks, as tolerated by the patient.

The recommended dose and schedule for Ranolazine is 500 mg twice daily. The dose is increased to 1000 mg twice daily, as needed, based on clinical symptoms. The composition recited hereinabove is described in U.S. Pat. Nos. 6,303,607; 6,369,062; 6,479,496; 6,503,911; 6,525,057; 6,562,826; 6,617,328; 6,620,814; 6,852,724; and 6,864,258, the entire contents of which are incorporated by reference.

The recommended dose and schedule for Bosentan is 62.5 mg twice daily for patients >12 years of age. After 4 weeks, the dose is increased to 125 mg twice daily if the patient weighs greater than 40 kg, and the dose is not changed if the patient weights less than 40 kg. The composition recited hereinabove is described in U.S. Pat. Nos. 7,959,945 and 8,309,126, the entire contents of which are incorporated by reference.

The recommended dose and schedule for Ambrisentan is 5 mg orally once a day. The dose is increased to 10 mg orally once a day, if 5 mg is tolerated by the patient. The composition recited hereinabove is described in U.S. Pat. Nos. 8,377,933; 9,474,752; and 9,549,926, the entire contents of which are incorporated by reference.

The recommended dose and schedule for Trepostinil is 0.25 mg orally every 12 hours or 0.125 mg every 8 hours for oral extended-release tablets; 3 breaths (18 mcg) per treatment session 4 times per day or if not tolerated then reduce to 1 or 2 breaths and subsequently increase to 3 breaths as tolerated for inhalation; or 1.25 ng/kg/min via continuous subcutaneous or IV infusion or 0.625 ng/kg/min if the larger dose cannot be tolerated for patients new to prostacyclin infusion therapy. The composition recited hereinabove is described in U.S. Pat. Nos. 10,076,505; 7,999,007; 8,653,137; 8,658,694; 9,199,908; 9,593,066; 9,604,901; and 9,713,599, the entire contents of which are incorporated by reference.

The recommended dose and schedule for Selexipag is 200 mcg orally twice a day. The dose is incrementally increased by 200 mcg orally twice a day at weekly intervals to the highest tolerated dose, not to exceed 1600 mcg orally twice a day. The composition recited hereinabove is described in U.S. Pat. Nos. 7,205,302; 8,791,122; 9,173,881; and 9,284,280, the entire contents of which are incorporated by reference.

The recommended dose and schedule for Ralinepag is 10 μg twice daily to 300 μg twice daily. The composition recited hereinabove is described in Efficacy and safety of ralinepag, a novel oral IP agonist, in PAH patients on mono or dual background therapy: results from a phase 2 randomised, parallel group, placebo-controlled trial (Torres et al. 2019), the entire contents of which are incorporated by reference.

The recommended dose and schedule for Riociguat is 1 mg orally 3 times a day. This dose is increased as tolerated, but is not to exceed 2.5 mg orally 3 times a day. The composition recited hereinabove is described in U.S. Pat. Nos. 6,743,798 and 7,173,037, the entire contents of which are incorporated by reference.

The recommended dose and schedule for Trimetazidine is 60 mg/day to 140 mg/day. The composition recited hereinabove is described in Defining the Role of Trimetazidine in the Treatment of Cardiovascular Disorders: Some Insights on Its Role in Heart Failure and Peripheral Artery Disease (Chrusciel et al. 2014), the entire contents of which are incorporated by reference.

The recommended dose and schedule for Sampatrilat is 50 mg to 100 mg daily. The composition recited hereinabove is described in Sustained Antihypertensive Actions of a Dual Angiotensin-Converting Enzyme Neutral Endopeptidase Inhibitor, Sampatrilat, in Black Hypertensive Subjects (Norton et al. 1999), the entire contents of which are incorporated by reference.

Gemopatrilat is described in Metabolism Of [14c] Gemopatrilat After Oral Administration To Rats, Dogs, And Humans (Wait et al. 2006), the entire contents of which are incorporated by reference.

The recommended dose and schedule for Fasidotril is 100 mg twice daily. The composition recited hereinabove is described in Antihypertensive effects of fasidotril, a dual inhibitor of neprilysin and angiotensin-converting enzyme, in rats and humans (Laurent et al. 2000), the entire contents of which are incorporated by reference.

The recommended dose and schedule for Omapatrilat is 10 mg to 80 mg daily. The composition recited hereinabove is described in Omapatrilat and enalapril in patients with hypertension: the Omapatrilat Cardiovascular Treatment vs. Enalapril (OCTAVE) trial (Kostis et al. 2004), the entire contents of which are incorporated by reference.

The recommended dose and schedule for Candoxatril is 200 mg twice a day to 400 mg twice a day. The composition recited hereinabove is described in Comparison of the short-term effects of candoxatril, an orally active neutral endopeptidase inhibitor, and frusemide in the treatment of patients with chronic heart failure (Northridge et al. 1999), the entire contents of which are incorporated by reference.

The recommended dose and schedule for Digoxin is 8 to 12 mcg/kg through intravenous administration for the total loading dose and increased to 0.1 to 0.4 mg/day for the maintenance regiment. For oral administration, the dose and schedule is 10 to 15 mcg/kg for the total loading dose and increased to 3.4 to 5.1 mcg/kg/day. Another dosing option is 0.125 to 0.25 mg per day for oral or intravenous administration, with higher doses of 0.375 to 0.5 mg/day rarely needed. The composition recited hereinabove is described in Digoxin: A systematic review in atrial fibrillation, congestive heart failure and post myocardial infarction (Virgadamo et al. 2015), the entire contents of which are incorporated by reference.

The recommended dose and schedule for Ivabradine is 5 mg orally twice a day with meals. This dose is increased as tolerated, but is not to exceed 7.5 mg orally twice a day. The composition recited hereinabove is described in U.S. Pat. Nos. 7,361,649; 7,361,650; 7,867,996; and 7,879,842, the entire contents of which are incorporated by reference.

The recommended dose and schedule for Hydralazine is 10 mg orally 4 times a day for the first 2 to 4 days, increased to 25 mg orally 4 times a day for the balance of the first week. This dose is increased to 50 mg orally 4 times a day for week 2 and subsequent weeks. The composition recited hereinabove is described in U.S. Pat. Nos. 6,465,463 and 6,784,177, the entire contents of which are incorporated by reference.

The recommended dose and schedule for Seralaxin is three 48-hour intravenous infusions of 30 μg/kg/day. The composition recited hereinabove is described in RELAX-REPEAT: A Multicenter, Prospective, Randomized, Double-Blind Study Evaluating the Safety and Tolerability of Repeat Doses of Serelaxin in Patients with Chronic Heart Failure (Teerlink et al. 2016), the entire contents of which are incorporated by reference.

The recommended dose and schedule for Nesiritide is 2 mcg/kg IV bolus, followed by 0.01 mcg/kg/min via continuous IV infusion; not to be titrated more frequently than every 3 hours to a maximum of 0.03 mcg/kg/min. The composition recited hereinabove is described in U.S. Pat. No. 5,114,923, the entire contents of which are incorporated by reference.

A subset of second agents that have a beneficial effect in a combination therapy with levosimendan include: K-ATP channel activators (e.g. pinacidil, diazoxide, bimakalim, levocromakalim, cromakalim, rimakalim, and nicorandil, etc.); nitrates (e.g. nitroglycerin-NTG, isosorbide dinitrate, etc.); nitrites (e.g. sodium nitrite, amyl nitrite, etc.); NO donors- (Sodium nitroprusside, Nitric Oxide, Molsidomine, linsidomine); PDE inhibitors (e.g. Milrinone, Pimobendan, Enoximone, etc.); natriuretic peptides, such as BNP (e.g. nesiritide), ANP (e.g. carparetide and ularitide), CDNP (e.g. cenderitide), and others (e.g. CNP, DNP, MANP, etc.); NEP inhibitors (e.g. sacubitril, sampatrilat/sympatril, fasidotril, omapatrilat/omapatril, candoxatril, etc.); and ARNIs (Entresto). Furthermore, a combination therapy with levosimendan may include any of the above second agents, a diuretic, or both.

Additionally, it is noted that combined pre and post capillary pulmonary hypertension and heart failure with preserved ejection fraction (Cpc-PH-HFpEF) is a small and special phenotype of certain PH-HFpEF patients. These patients may benefit from agents that reduce pulmonary vascular resistance (Opitz 2016). The HELP Study identified that Levosimendan did not decrease pulmonary vascular resistance, particularly upon chronic administration. Therefore, Cpc-PH-HFpEF patients may benefit from a combination therapy comprising Levosimendan and an agent that reduces pulmonary vascular resistance. Accordingly, a combination therapy of Levosimendan with a pulmonary vasodilator, including but not limited to, phosphodiesterase-5 inhibitors (PDE-5 inhibitors, e.g. sildenafil, tadalafil, etc.); endothelin receptor antagonists (ERAs, e.g. bosentan, ambrisentan, etc.); and prostacyclins (e.g. epoprostenol, iloprost, Treprostinil, etc.) may provide therapeutic benefits to Cpc-PH-HFpEF patients.

Each drug can be administered in the dose and regiment that has been disclosed in the drug's aforementioned literature.

The embodiments referred to above refer to several drugs being substantially effective in the body at a same time. Several drugs can be administered substantially at the same time, or can be administered at different times but have effect on the body at the same time. For example, this includes administering levosimendan before or subsequently, while functioning oflevosimendan in the body is substantially extant.

Therefore, the state of the art at the time of filing is that the effects of combination therapy of two drugs, in particular levosimendan and a second agent, cannot be predicted until the results of combination studies are available.

Each embodiment disclosed herein is contemplated as being applicable to each of the other disclosed embodiments. Thus, all combinations of the various elements described herein are within the scope of the invention.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

Accordingly, the following examples are presented to more fully illustrate some embodiments of the invention. They should, in no way be construed, however, as limiting the broad scope of the invention.

### EXAMPLES

### Example 1

### Brief Summary of HELP Study Open Label Extension Study

The initial HELP Study is described in PCT International Application Publication No. WO 2021/126884 and Burkhoff, et al. (2021) “Levosimendan improves hemodynamics and exercise tolerance in PH-HFpEF: results of the randomized placebo-controlled HELP trial,” the entire contents of each of which are incorporated by reference. As an open label extension to the HELP study, patients were transitioned from weekly I.V. levosimendan administration to daily oral levosimendan administration, which has the benefits of more stable dosing, less risk of infection, and convenience. Among the goals of the extension study was to determine the dose of oral levosimendan that could maintain the efficacy of the weekly I.V. infusions of levosimendan.

### Details of HELP Study Open Label Extension Study

PH-HFpEF is a progressive, fatal disease for which there are no approved therapies. All patients that the 6-week parent TNX-LVO-04 study (also referred to as the initial Help Study) underwent assessments of safety and efficacy in a double-blind randomized protocol. When the trial was completed, the data supported that levosimendan was not only safe, but also effective as shown in the hemodynamic changes at rest and with exercise and further by the clinical improvement in exercise capacity demonstrated in in the improvement in 6-minute walk. No other treatment has ever been shown to be safe and effective in patients with PH-HFpEF. For these reasons, all participating patients were allowed to enter an open-label extension study (also referred to as TNX-LVO-05), to provide ongoing levosimendan therapy for two additional years.

The efficacy of the once weekly levosimendan regimen is supported by an active metabolite with an extended half-life of 70-80 hours. The metabolite peaks approximately two days after completing the weekly dose, declining through the remainder of the week. Data from the parent TNX-LVO-04 study indicate patients' response to therapy peaks early in the week, followed by a decline in keeping with exposure to the active metabolite. Patients that entered TNX-LVO-05 were required to undergo the insertion of a port-a-cath, learn how to self-administer the levosimendan infusion through a port-a-cath at home, and weekly administer IV levosimendan through their port-a-cath for 24 hours. It is important to note that patients who entered TNX-LVO-05 have chosen to continue their 24-hour weekly levosimendan dose regimen, acknowledging that patients recognize the benefits of the drug.

As an amendment to the initial TNX-LVO-05 protocol, the FDA agreed to allow ongoing TNX-LVO-05 patients to transition from a weekly 24-hour I.V. levosimendan administration to an oral levosimendan administration.

Notably, these PH-HFpEF patients have no therapeutic alternatives for treatment, and the oral formulation of levosimendan offers several potential benefits to these patients. From previous studies, such those in heart failure and ALS patients, a daily oral regimen allows patients to maintain peak levels of active drug, maximizing the effectiveness of their levosimendan regimen.

Furthermore, the oral formulation offers better safety. The weekly IV administration through a port-a-cath for 24 hours carries risks of infection and thrombosis.

Thus, the purpose of the TNX-LVO-05 study is unchanged, as the study was designed to allow PH-HFpEF patients that have no therapeutic alternatives for treatment to receive therapy with levosimendan to which they have shown a hemodynamic response. This is even more justified based on the clinical benefits demonstrated on exercise capacity.

The population to be enrolled in TNX-LVO-05 is identical. Only patients who are currently enrolled in the open label extension study could be included.

The procedures included in TNX-LVO-05 were changed only with respect to determining the comparable oral dose of the existing IV formulation. Once that dose is determined, the study is identical with respect to the protocol. Since the I.V. formulation is weight based, and bypasses issues of bioavailability that may be affected by drug absorption, a dose escalation strategy is being adopted, guided by the individual patient response and any adverse events that may occur. Thus, the measurement of a six-minute walk test and patient questionnaire before and after the transition was to ensure that the patient continues to benefit. In addition, the monitoring of the patients' blood pressure and heart rate each day was to ensure that the oral dose remains safe.

There has been no requirement from the FDA to transition these patients from the I.V. formulation to the oral formulation. Rather, the transition was decided on for humanitarian reasons. Also, to the potential benefit of the patients, these patients were offered the option of switching from an IV to an oral route. In addition, for humanitarian reasons, to the protocol also extends the period which levosimendan will be made available for an additional three years. The fact that PH-HFpEF is a progressive and fatal disease with no other potential treatments was paramount to this decision.

Patients who declined to switch to the oral formulation were allowed to continue with the IV formulation until the end of the two years. Patients who did not tolerate the oral formulation were allowed to resume the IV formulation until the end of the two years.

The following considerations justified amending the TNX-LVO-05 study protocol:
1. The amended TNX-LVO-05 protocol included the same patients enrolled in the original study. This was not a new study of the patients currently enrolled. This was open label, with no blinding of patients or physicians, and no control group.
2. The scope and purpose of the study was unchanged, with the exception that it offered the further extension of treatment allowed by the transition to oral therapy (See item #4).
3. The amendment clearly delineated a stepwise and thoughtful titration of patients from their intravenous to oral levosimendan to ensure the safe and effective transition of their levosimendan regimen.
   a. Patients were started on 1 mg daily, representing a dose generally less than half their total weekly weight-based dose regimen.
   b. Patients were titrated based on tolerance to the daily levosimendan paying attention to their symptoms of PH-HFpEF, and special attention to specific adverse events of interest, heart rate and blood pressure. A daily diary captured patient vitals and symptoms to aid their physician in titrating patients at two-week intervals.
   c. Patient response also was evaluated by clinical measures to provide the physician with additional information on the patient's response.
   d. While most patients were expected to receive 2-3 mg daily at the conclusion of the transition study, the dose escalation strategy was included to allow all patients to realize the same benefit as they did from the I.V. formulation.
   e. To document the sustained efficacy of the oral formulation in these patients, patients had to undergo a six-minute walk test and patient questionnaire prior to and at the end of the transition study.
4. The transition to oral levosimendan allowed for extension of the study from its initial two years to an additional three years. This offers these PH-HFpEF patients, with no other treatments available, to continue on a drug that they have responded to.
5. The amendment required patients to sign a revised informed consent to ensure patients are completely informed concerning the transition amendment. Patients that preferred to continue with their weekly 24-hour I.V. regimen were allowed to continue through the initial two-year period of the study and then will be withdrawn.

### Safety Review of HELP Study Open Label Extension Study

This is the third safety review for Study Protocol No. TNX-LVO-05. This review a) summarizes treatment emergent adverse events (TEAEs), treatment emergent serious adverse events (TESAEs), discontinuations due to TEAEs, and b) provides an overall assessment of safety data.

As of 4 Nov. 2020, 36 subjects have been enrolled in this open-label study with levosimendan and 18 (−5; this number presents change versus previous Safety Review) are still active. Seven subjects (+3) withdrew due to AE, and eight (+2) subjects withdrew consent. One subject was withdrawn by investigator decision.

### Treatment Emergent Adverse Events (TEAEs)

### 1. Overview

Of the 36 subjects enrolled, TEAEs were reported in 32 subjects (88.9%)(+1; this number presents change versus previous Safety Review); 20 subjects (55.6%)(+3) experienced serious TEAEs, of which two were considered related (5.6%) (+1). Five subjects (2.9%)(+1) permanently discontinued study drug due to TEAEs.

Of the 32 subjects enrolled who experienced TEAEs, severity was considered as mild in 10 subjects (27.8%)(−1), moderate in 9 subjects (25.0%)(+1), and severe in 13 subjects (36.1%)(+1).

The highest number of subjects with TEAEs was reported under the SOC of Cardiac disorders, and Infections and Infestations, each with 17 subjects (47.2%)(+5) followed by General disorders and administration site conditions and Nervous system disorders, each with 15 subjects (41.736.1%)(+2)

TEAEs reported in 3 or more subjects include: dizziness (8 subjects), dyspnea (8 subjects), hypokalemia (7 subjects), cardiac failure (6 subjects), headache (5 subjects), palpitations (4), ventricular extrasystoles (3), pneumonia (3), upper respiratory tract infection (3 subjects), urinary tract infection (3), asthenia (3), fluid overload (3), gout (3 subjects), hemoptysis (3), dermatitis (3 subjects), arthralgia (3 subjects), back pain (3 subjects), device infusion issues (3 subjects), and weight increased (3 subjects).

The emerging TEAE profile is concurrent with the incidence expected in the studied population.

### 2. Related TEAEs

TEAEs that were considered related to the study drug occurred in 15 subjects (41.7%)(+2). The highest number of subjects with related TEAEs was reported under the SOC of cardiac disorders with 5 subjects (8.3%)(+2), followed by SOC of Product issues with 4 subjects (11.4%), SOC of Nervous system disorders with 3 subjects (8.3%), SOC of Respiratory, thoracic and mediastinal disorders with 3 subjects (8.3%), and SOC of Skin and subcutaneous tissue disorders with 3 subjects (8.3%).

Two related TEAEs were reported as serious (angioedema and hyponatremia); all others were considered not serious.

Drug-related TEAEs reported in 2 or more subjects include: device infusion issue (3 subjects), palpitations (2), ventricular extrasystoles (2), headache (2), and urticaria (2 subjects).

### 3. Treatment Emergent Serious Adverse Events (TESAEs)

Of the 36 subjects enrolled, TESAEs occurred in 20 subjects (55.6%)(+3). Except for two (hyponatremia and angioedema), all TESAEs were considered not related to the study drug.

The highest number of subjects with TESAEs was reported under the SOC of cardiac disorders with 8 subjects (22.2%)(+2) and in Infections and Infestations with 8 subjects (22.2%)(+2), followed by Metabolism and nutrition disorders with 4 subjects (8.3%)(+1).

Cardiac failure was reported as TESAE in 4 subjects (11.1%), cardiac failure congestive in 2 subjects (5.6%), pneumonia in 3 subjects (8.3%), and asthenia in 2 subjects (5.6%). All other TESAE's were reported in only a single subject.

4. TEAEs that LED to Study Drug Discontinuation

Of the 36 subjects enrolled, treatment with the study drug was permanently discontinued due to a TEAE in 5 subjects (13.9%)(+1).

One subject (021-008) reported ageneralized rash and allergic reaction, starting on day 14 of the study. The rash and allergic reaction were considered of moderate intensity and related to study drug. The rash subsided by day 20 of the study, but the allergic reaction persisted. Study drug was permanently withdrawn.

One subject (011-003) reported acute cardiac failure, starting on day 487 of the study. The cardiac decompensation was considered of severe intensity, serious in nature, and not related to study drug. Study drug was permanently withdrawn.

One subject (021-003) died of unknown cause on day 334 of the study. The death was considered of severe intensity, serious in nature, and not related to study drug. Study drug was permanently withdrawn. This subject reported also TESAE ofhypokalemia, metastatic brain adenocarcinoma, generalized weakness and headache, and type 2 diabetes mellitus.

One subject (017-010) reported vascular access site infection, starting on day 150 of the study. The vascular access site infection was considered of severe intensity, serious in nature, and not related to study drug. The infection was considered recovered by day 153. Study drug was permanently withdrawn.

One subject (009-007) reported worsening of heart failure, starting on day 393 of the study. The worsening heart failure considered of severe intensity, serious in nature, and not related to study drug. Study drug was permanently withdrawn.

### 5. Overall TEAE Assessment

In this open label study with levosimendan in subjects with pulmonary hypertension with heart failure and preserved ejection fraction, the adverse event profile is concurrent with the underlying disease state.

Of note, is one case of angioedema, and another subject who discontinued study drug due to allergic reaction.

### Overall Safety Assessment

There are no safety trends or signals noted in this data review.

### Summary of Extension Study Results for Patients Who Transitioned from I.V. To Oral Levosimendan

Eighteen (18) patients with PH-HFpEF who had received I.V. levosimendan administration for two years were safely transitioned to oral levosimendan administration over a 6-8 week period. Fifteen (15) patients responded best to 3 mg/day in divided doses.

Three (3) patients responded best to 4 mg/day in divided doses.

Furthermore, oral levosimendan administration produced improvements in (1) six minute walk distance by 32%; (2) BNP/NT-ProBNP biomarker measurements by 22%; and (3) patient reported outcomes in a questionnaire (KCCQ) compared to PH-HFpEF patients who had been receiving weekly I.V. infusion.

Lastly, no SAEs were reported that were attributed to the study drug. Notably, two patients with increased heart rate at a final clinic visit that were inconsistent with their home diaries.

TABLE A
Summary 6MW Change (Baseline vs Week 6/8) Result for Patients
who Transitioned from I.V. to Oral Levosimendan

| Patient # | 6MW Change |
|---|---|
| 004001 | 30 |
| 004002 | 30 |
| 007002 | −1 |
| 009001 | 30 |
| 009003 | −15 |
| 009008 | 78 |
| 009010 | −27 |
| 017001 | −10 |
| 019002 | −31 |
| 019004 | −4 |
| 019007 | 24 |
| 021001 | −23 |
| 021002 | −112 |
| 021007 | 9 |
| 021009 | −17 |

TABLE A2
Summary 6MW Change (BL vs Week 6 only) Result for Patients
who Transitioned from I.V. to Oral Levosimendan

| Patient # | 6MW Change |
|---|---|
| 004001 | 30 |
| 004002 | 15 |
| 007002 | −1 |
| 009001 | 30 |
| 009003 | −137 |
| 009008 | 78 |
| 009010 | −27 |
| 010003 | 118 |
| 017001 | −10 |
| 019004 | −4 |
| 019007 | 5 |
| 021002 | −9 |
| 021-005 | −8 |
| 021007 | 9 |
| 021009 | −17 |
| 022002 | 40 |

The final statistical analysis for change in 6MWD at week 6 was as follows:

Week 6 - Oral

| n | 16 |
|---|---|
| Mean | 302.0 |
| Standard deviation | 119.84 |
| Minimum | 91 |
| Median | 292.0 |
| Maximum | 550 |

Change from baseline to Week 6 - Oral [2]

| n | 16 |
|---|---|
| Mean | 7.0 |
| Standard deviation | 53.54 |
| Minimum | −137 |
| Median | 2.0 |
| Maximum | 118 |

TABLE B
Results for Patients who Transitioned from I.V.
to Oral Levosimendan - (Baseline to Week 24-48)

| | HELP | | | | | | | | |
|---|---|---|---|---|---|---|---|---|---|
| | Study | HELP | | HELP | | TNX-LVO-05 | | TNX-LVO-05 | |
| | Active/ | (BL) | | (WK 6) | | (BL) | | (WK 24/48) | |
| Patient # | Placebo | 6MWT | HR | 6MWT | HR | 6MWT | HR | 6MWT | HR |
| 004-001 | Active | 484 | 60 | 482 | 75 | 502 | 70 | 590 | 91 |
| 004-002 | Placebo | 260 | 61 | 230 | 60 | 295 | 64 | 310 | 62 |
| 007-002 | Placebo | 188 | 71 | 216 | 73 | 232 | 76 | 180 | 64 |
| 009-001 | Placebo | 329 | 62 | 329 | 70 | 320 | 62 | 312 | 62 |
| 009-003 | Placebo | 273 | 47 | 171 | 50 | 198 | 63 | 255 | 62 |
| 009-008 | Placebo | 358 | 88 | 350 | 70 | 335 | 87 | ND | 85 |
| 009-010 | Placebo | 305 | 62 | ND | 68 | 274 | 59 | ND | 70 |
| 010-003 | Active | 366 | 77 | 311 | 72 | 311 | 78 | 347 | 69 |
| 017-001 | Active | 364 | 63 | 397 | 63 | 375 | 67 | 393 | 74 |
| 019-002 | Placebo | 238 | 66 | 234 | 53 | 224 | 71 | 313 | 58 |
| 019-004 | Placebo | 142 | 95 | 60 | 70 | 162 | 70 | 91 | 73 |
| 019-007 | Active | 295 | 73 | 270 | 86 | 360 | 82 | 274 | 82 |
| 021-001 | Active | 211 | 70 | 218 | 71 | 220 | 84 | 213 | 75 |
| 021-002 | Placebo | 352 | 79 | 365 | 71 | 368 | 83 | ND | 87 |
| 021-005 | Active | 228 | 77 | 232 | 63 | 232 | 77 | 225 | 65 |
| 021-007 | Active | 367 | 65 | 401 | 67 | 401 | 68 | ND | 78 |
| 021-009 | Active | 299 | 73 | 196 | 75 | 196 | 72 | ND | 77 |
| 022-002 | Placebo | 231 | 70 | 236 | 67 | 205 | 87 | 174 | 85 |

TABLE C
Results for Patients who Transitioned from I.V. to Oral Levosimendan

| | | | | | | | Week 0 to | Week 0 to | |
|---|---|---|---|---|---|---|---|---|---|
| | | | | | | | Week 6/8 | Week 6/8 | Dose at |
| | Week 0 | | Week 6 | | Week 8 | | 6MW | HR | Week 6/8 |
| Patient # | 6MWT | HR | 6MWT | HR | 6MWT | HR | Change | Change | TID/QID |
| 004-001 | 520 | 73 | 550 | 90 | NA | NA | 30 | 17 | TID |
| 004-002 | 280 | 59 | 295 | 94 | 310 | 63 | 30 | 35 | QID |
| 007-002 | 202 | 80 | 201 | 71 | NA | NA | −1 | −9 | TID |
| 009-001 | 259 | 60 | 289 | 59 | NA | NA | 30 | −1 | TID |
| 009-003 | 228 | 59 | 91 | 57 | 213 | 64 | −15 | −2 | QID |
| 009-008 | 318 | 77 | 396 | 81 | NA | NA | 78 | 4 | TID |
| 009-010 | 408 | 63 | 381 | 73 | 381 | 82 | −27 | 10 | TID |
| 010-003 | 340 | 68 | 458 | 76 | NA | NA | 118 | 8 | TID |
| 017-001 | 375 | 78 | 365 | 83 | NA | NA | −10 | 5 | TID |
| 019-002 | 265 | 56 | ND | ND | 234 | 75 | −31 | 19 | TID |
| 019-004 | 175 | 73 | 171 | 70 | NA | NA | −4 | −3 | TID |
| 019-007 | 249 | 73 | 273 | 82 | NA | NA | 24 | 9 | TID |
| 021-001 | 224 | 64 | ND | 70 | 201 | 70 | −23 | 6 | TID |
| 021-002 | 341 | 72 | 332 | 65 | 229 | 84 | −112 | −7 | QID |
| 021-005 | 182 | 106 | 174 | 104 | NA | NA | −8 | −2 | TID |
| 021-007 | 388 | 77 | 397 | 87 | NA | NA | 9 | 10 | TID |
| 021-009 | 266 | 91 | 249 | 93 | NA | NA | −17 | 2 | TID |
| 022-002 | 170 | 80 | 210 | 91 | 159 | 60 | 40 | 11 | TID |

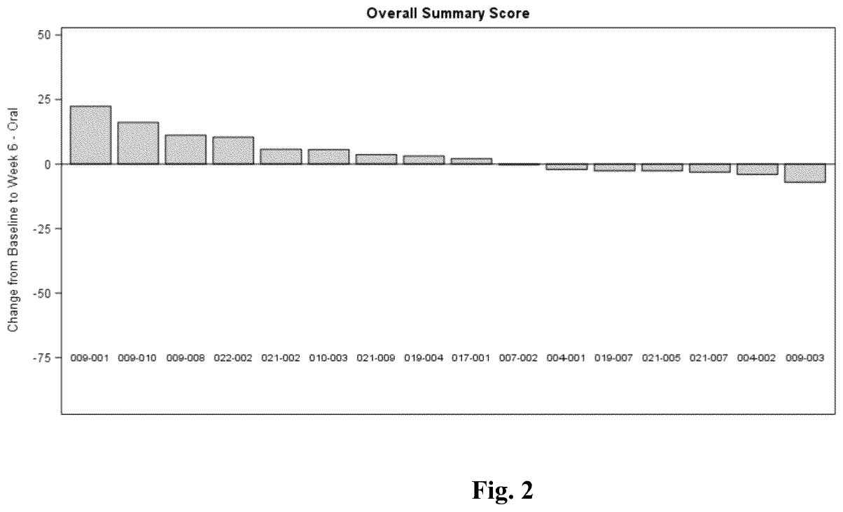

Additional KCCQ results for patients who transitioned from I.V. to oral levosimendan are shown in FIG. 2.

### Example 2

During the HELP Study, as described in PCT International Application Publication No. WO 2021/126884, the entire contents of which are incorporated by reference, Patient 019-002 was administered empagliflozin shortly after beginning levosimendan therapy.

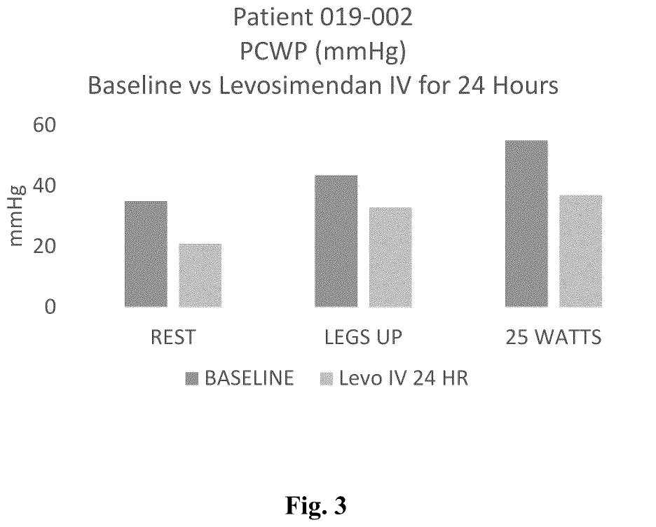

Specifically, Patient 019-002 had established PH-HFpEF and was initially enrolled into the HELP Trial. The diagnosis of PH-HFpEF was confirmed by rest and exercise right heart catheterization. At baseline, the patient had a PCWP at rest of 35 mmHg, and a pulmonary artery pressure of 79/32 mmHg. The PCWP of 35 mmHg at rest increased with exercise to 55 mmHg at baseline. Following 24 hours of i.v. levosimendan, Patient 019-002 had a PCWP at rest of 21 mmHg, which increased with exercise to 37 mmHg. Thus, levosimendan produced a 40% fall in resting PCWP and a 33% fall in exercise PCWP. In addition, at baseline the CVP was 18 mmHg and increased to 30 mmHg with exercise. Following the 24 hours of i.v. levosimendan the CVP fell to 12 mmHg at rest and to 21 mmHg with exercise. Thus, levosimendan produced a 33% fall in resting CVP, and a 30% fall in CVP with exercise. Accordingly, the response of Patient 019-002 to levosimendan treatment was typical of other patients in the study in terms of improvements to their hemodynamic profile (FIG. 3).

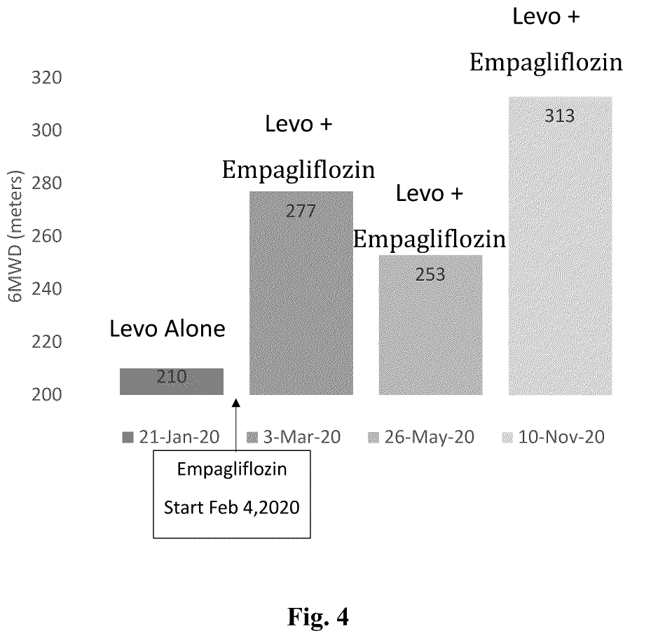

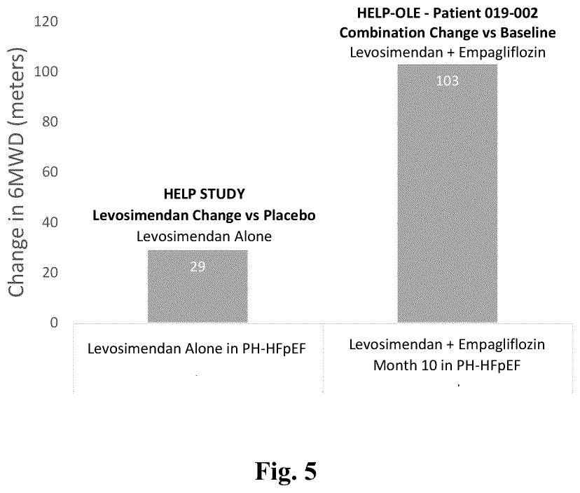

However, the patient showed an unexpectedly dramatic increase in their 6-minute walk distance (6MWD) upon receiving the combination of levosimendan and empagliflozin (FIG. 4). While the HELP Study found that levosimendan alone improved the 6-minute walk distance of PH-HFpEF patients by approximately 29 m on average, Patient 019-002 increased their 6MWD by over 100 m when receiving a combination of levosimendan and empagliflozin (FIG. 5). Previous clinical trials that studied treating HFpEF, HFrEF, or PH-HFpEF patients with empagliflozin alone (e.g. EMPERIAL-Preserved, EMPERIAL-Reduced, (Abraham et al.) and EMBRACE HF (Nassif et al.)) showed that the patients did not demonstrate a material change in their exercise capacity as measured by a difference in their 6MWD. Accordingly, the combination of levosimendan and empagliflozin showed a greater than expected synergistic effect on improving exercise capacity when administered as a heart failure treatment. These results suggest that effective treatment of heart failure, with or without accompanying pulmonary hypertension, can be achieved by a combination therapy of levosimendan and an SGLT-2 inhibitor.

In view of the oral extension study discussed in Example 1, the combination of orally administered levosimendan and an SGLT-2 inhibitor works analogously to the data as shown for a combination of I.V. administered levosimendan and an SGLT-2 inhibitor.

### Example 3

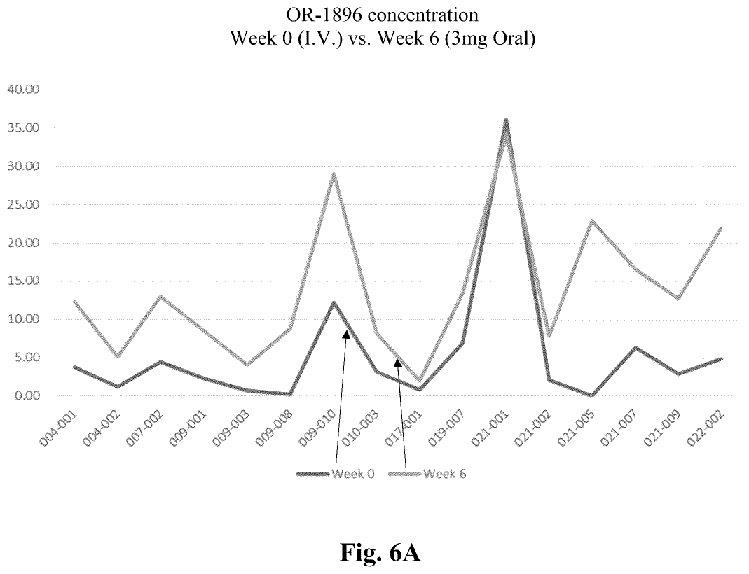

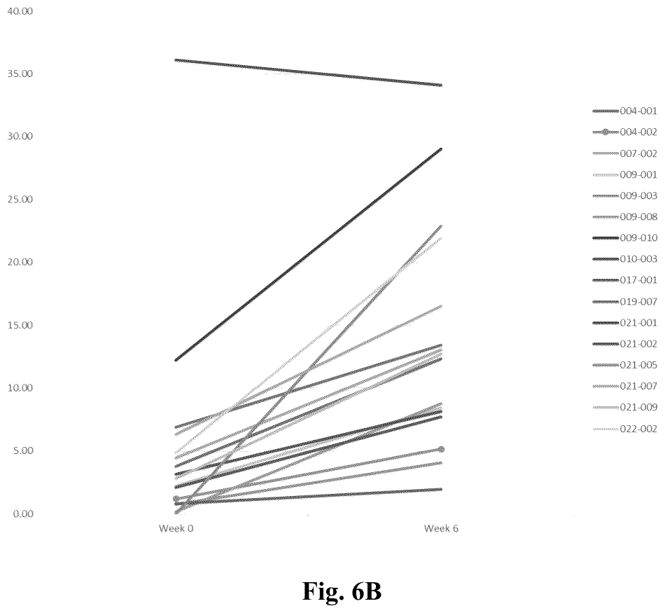

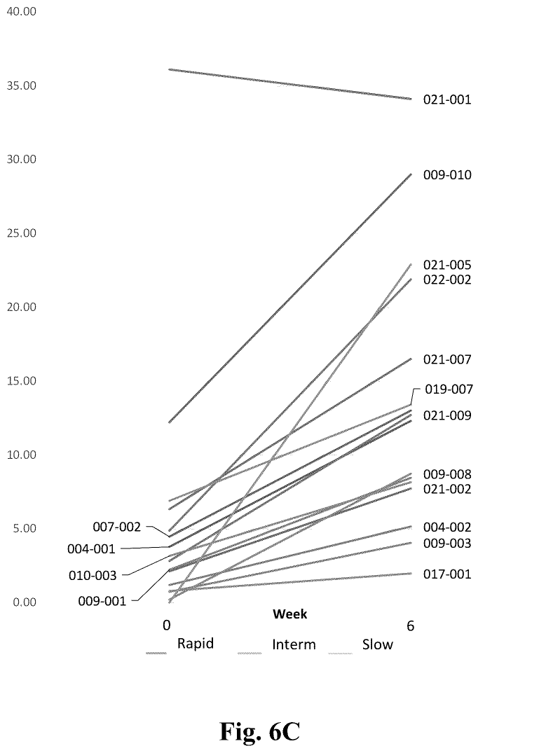

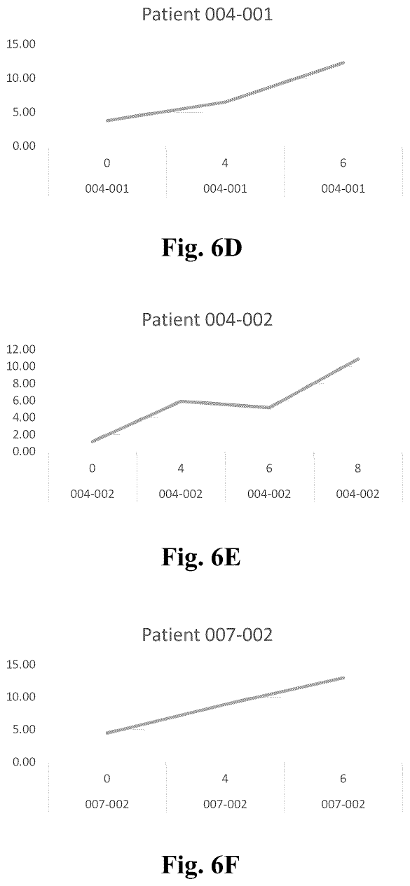

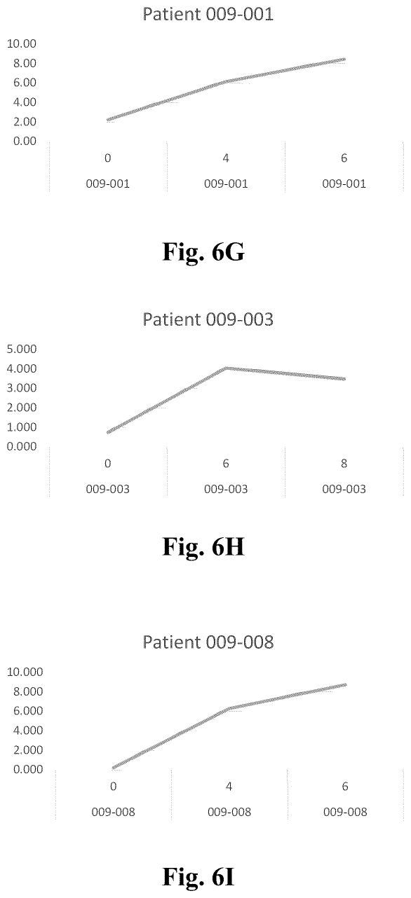

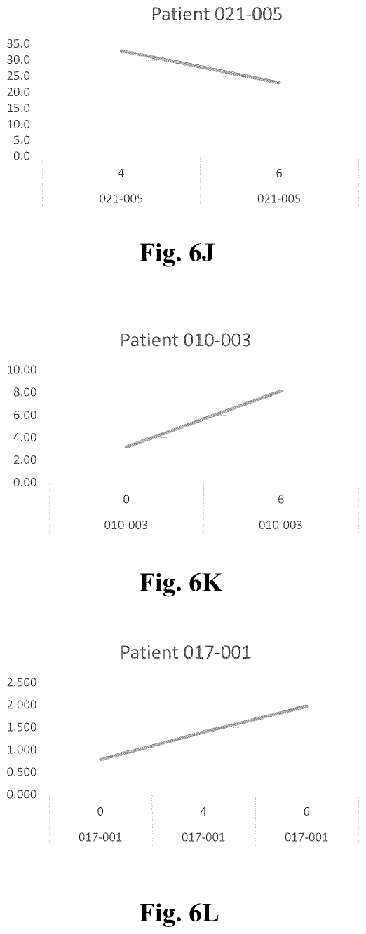

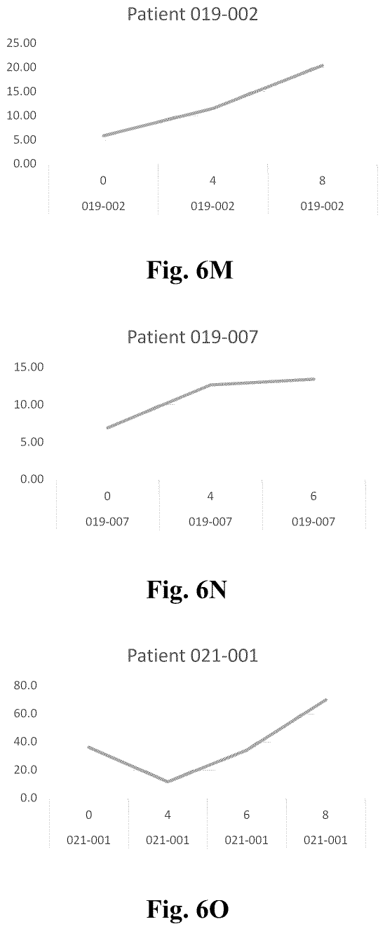

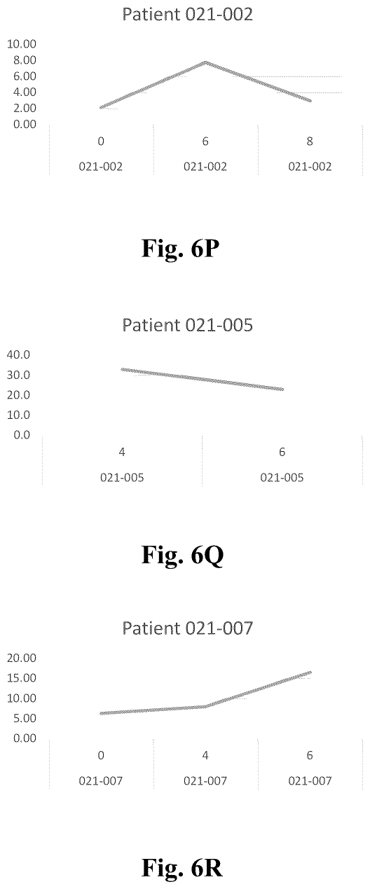

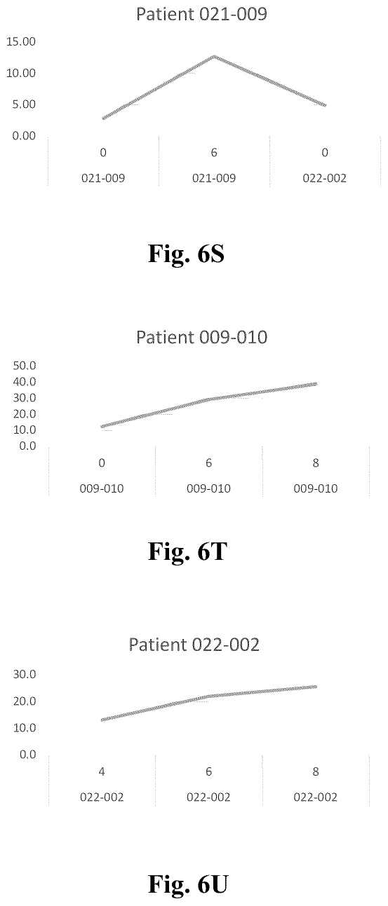

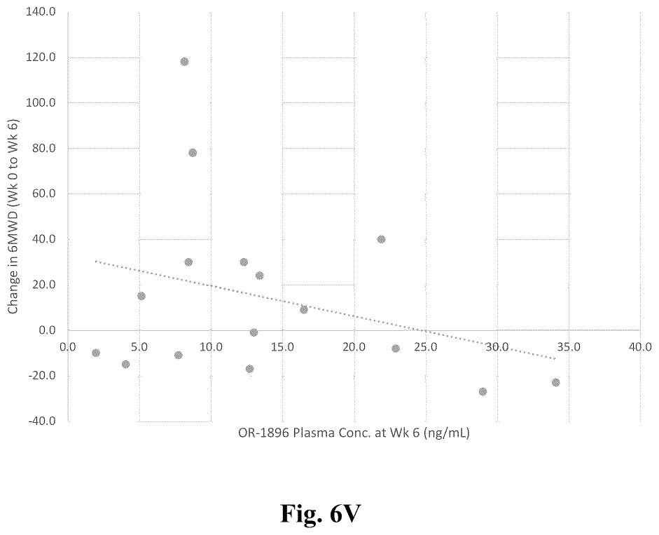

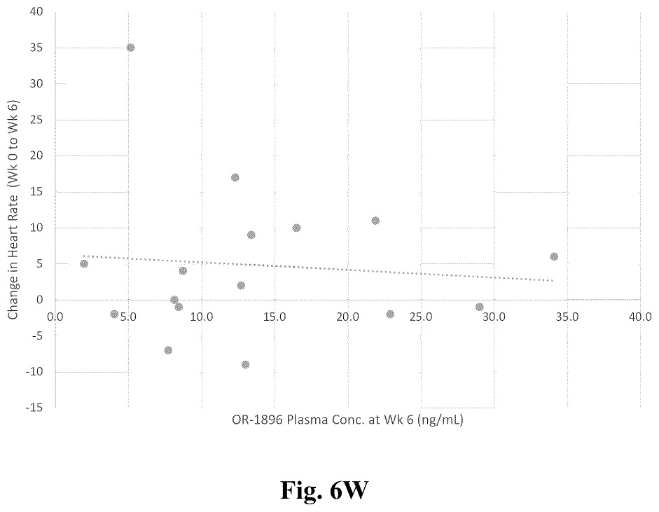

Pharmacokinetic data demonstrates that oral daily levosimendan dosing provides at least as good blood levels of OR-1896, an active levosimendan metabolite, and in most cases higher blood levels, than the weekly I.V. dosing. See Table C below and FIGS. 6A-6W.

TABLE C
Individual Patient Data from I.V. to Oral Transition Pharmacokinetic
Study showing OR-1896 Plasma concentration (ng/ml) by week and
acetylation Status

| | | | | | Acetylation |
|---|---|---|---|---|---|
| Patient # | Week 0 | Week 4 | Week 6 | Week 8 | Status |
| 004-001 | 3.77 | 6.50 | 12.3 | No data | I |
| 004-002 | 1.19 | 5.90 | 5.15 | 10.9 | S |
| 007-002 | 4.46 | 8.86 | 13.0 | No data | I |
| 009-001 | 2.22 | 6.14 | 8.44 | No data | S |
| 009-003 | 0.730 | No data | 4.04 | 3.47 | S |
| 009-008 | 0.197 | 6.28 | 8.73 | No data | S |
| 009-010 | 12.2 | No data | 29.0 | 38.7 | I |
| 010-003 | 3.16 | No data | 8.15 | No data | S |
| 017-001 | 0.787 | 1.39 | 1.97 | No data | S |
| 019-007 | 6.89 | 12.6 | 13.4 | No data | S |
| 021-001 | 36.1 | 11.6 | 34.1 | 69.9 | R |
| 021-002 | 2.11 | No data | 7.72 | 2.93 | I |
| 021-005 | 0.00 | 32.9 | 22.9 | No data | S |
| 021-007 | 6.32 | 7.96 | 16.5 | No data | I |
| 021-009 | 2.81 | No data | 12.7 | 25.5 | I |
| 022-002 | 4.86 | 13.1 | 21.9 | 25.5 | I |

### Example 4

Introduction: The HELP Trial demonstrated that once a week intravenous (IV) levosimendan infusions improve hemodynamics and exercise capacity in patients with pulmonary hypertension with heart failure and preserved ejection fraction (PH-HFpEF). However, a daily oral formulation would offer an advantage of stable dosing and eliminate the risk of line infections and thrombosis.

Hypothesis: Patients with PH-HFpEF who are receiving chronic IV levosimendan can be transitioned to oral levosimendan safely without sacrificing efficacy.

Methods: Patients with PH-HFpEF who were in the open label extension study of the HELP Trial for more than 18 months volunteered to enroll in this transition protocol. Patients were initiated with 1 mg/day of oral levosimendan 5-7 days after the last IV levosimendan infusion. The oral dose was increased every 2 weeks by 1 mg/day to a maximum of 4 mg/day over an 8 week period. The highest daily dose was determined by the investigator based on clinical assessments of symptoms and side effects. The primary safety endpoints were change in resting heart rate and blood pressure from baseline to final study visit and all other adverse events. Secondary efficacy endpoints included change in six-minute walk distance (6MWD), serum brain natriuretic peptide (BNP) or NT-proBNP levels, and quality of life as assessed by Kansas City Cardiomyopathy Questionnaire clinical summary score (KCCQ-CS) and overall summary score (KCCQ-OS) from baseline to final study visit. All comparisons were the baseline and at week 6-8 measurements at the highest daily dose of levosimendan.

Results: Eighteen patients (69±9 yrs, 72% women) participated in the transition study. The final dose of oral levosimendan in fifteen patients was 3 mg/day at 6 weeks, and in three patients was 4 mg/day at 8 weeks. One patient experienced a serious adverse event (SAE) unrelated to study drug and was discontinued. There were no SAEs related to oral levosimendan. The mean change in resting heart rate was 4.9 (SD±7.5) beats/min and mean systolic arterial blood pressure was 4.1 (SD±12.6) mm Hg. The mean change in 6MWD (n=17) was 13.1 (SD±39.5) meters, and 6MWD (n=16) was 7.0 (SD±53.5). See final summary statistical analysis of safety population.

Heart Rate Change from Baseline to Week 6 Oral

| n | 18 |
|---|---|
| Mean | 6.9 |
| Standard deviation | 11.79 |
| Minimum | −9 |
| Median | 5.5 |

SBP Change from baseline to Week 6 - Oral

| n | 18 |
|---|---|
| Mean | 4.1 |
| Standard deviation | 12.64 |
| Minimum | −15 |
| Median | 1.5 |
| Maximum | 34 |

6MWD Change from Baseline to Week 6 - Oral

| n | 16 |
|---|---|
| Mean | 302.0 |
| Standard deviation | 119.84 |
| Minimum | 91 |
| Median | 292.0 |
| Maximum | 550 |

Change from baseline to Week 6 - Oral

| n [2] | 16 |
|---|---|
| Mean | 7.0 |
| Standard deviation | 53.54 |
| Minimum | −137 |
| Median | 2.0 |
| Maximum | 118 |

BNP (n=8) was −133.3 (SD 136.6) pg/dl, and NT-proBNP (n=7) was −239.4 (SD 548.1) pg/dl. The mean KCCQ-TS, KCCQ-CS and KCCQ-OS score (n=16) improved by 4.7, 2.5 points and 3.7 points, respectively.

Conclusions: The transition to oral levosimendan was well tolerated without safety concerns over a 6-8-week period in patients with PH-HFpEF who had been receiving IV levosimendan for more than 18 months. Oral levosimendan was also associated with further improvements in 6MWD, BNP/NT-ProBNP, and KCCQ scores. Oral levosimendan at 3-4 mg/day appears to be a superior formulation for chronic use in PH-HFpEF.

Thus, the transition from chronic intravenous to oral levosimendan is safe and effective in patients with pulmonary hypertension with heart failure and preserved ejection fraction.

### Example 5

Purpose/Objective: The HELP Trial demonstrated that once a week intravenous (IV) levosimendan infusions improve hemodynamics and exercise capacity in patients with pulmonary hypertension with heart failure and preserved ejection fraction (PH-HFpEF). However, a daily oral formulation would offer an advantage of stable dosing and eliminate the risk of line infections and thrombosis.

Hypothesis: Patients with PH-HFpEF who are receiving chronic IV levosimendan can be transitioned to oral levosimendan safely without sacrificing efficacy.

Materials and Methods: Patients with PH-HFpEF who were in the open label extension study of the HELP Trial for more than 18 months volunteered to enroll in this transition protocol.

The oral dosing schedule is provided in the table below. The 6-week transition period runs from week 0 through week 6.

| Previous IV | Week 0 | Week 2 | Week 4 | Week 6 | Week 8 |
|---|---|---|---|---|---|
| Infusion | (Office) | (Home) | (Home) | (Office) | (Office) |
| 0.10 | 1 mg QD | 1 mg BID | 1 mg TID | Patient | ONLY patients titrated |
| μg/kg/min | (1 mg total daily | (2 mg total | (3 mg total | evaluated for | (up or down) at Week 6 |
| | dose) | daily dose) | daily dose) | further | No further dose titration |
| | Morning | Every 12 hrs. | Every 8 hrs. | titration | above 4 mg QID |
| | | | | (up or down) | |

### Primary and Secondary Measures:

### Primary Safety Measures:

Change in resting heart rate and blood pressure from baseline to final study visit
All other adverse events

### Secondary Efficacy Measures:

Change in six-minute walk distance (6MWD) from baseline to final study visit
Change in serum brain natriuretic peptide (BNP) or NT-proBNP levels from baseline to final study visit
Change in quality of life as assessed by Kansas City Cardiomyopathy Questionnaire clinical summary score (KCCQ-CS) and overall summary score (KCCQ-OS) from baseline to final study visit

All comparisons were the baseline and at week 6-8 measurements at the highest daily dose of levosimendan.

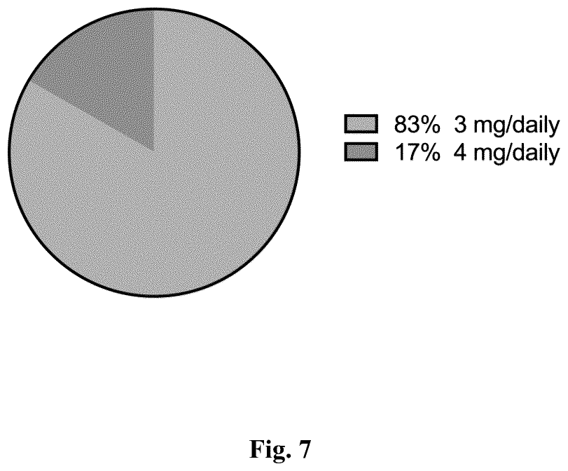

Results: Eighteen patients participated in the transition study. Mean Age: 69±9 years. Gender: 73% women. A final dosing chart is shown in FIG. 7.

### Primary Safety Measures

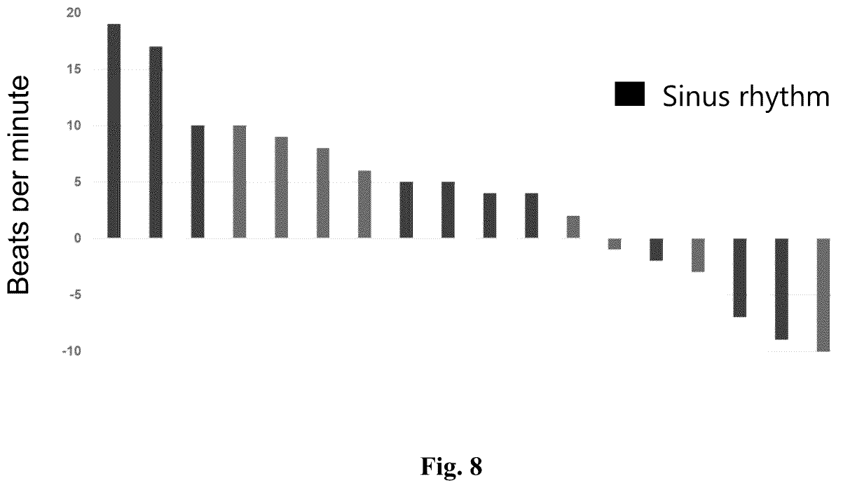

Heart Rate: See graph shown in FIG. 8

Blood Pressure: Mean change in systolic arterial blood pressure: 4.1 (SD±12.6) mm Hg

### Adverse Events:

One patient had a serious adverse event not related to the study drug—Adnexal cystic mass and septic shock.

No other adverse events.

### Secondary Efficacy Measures

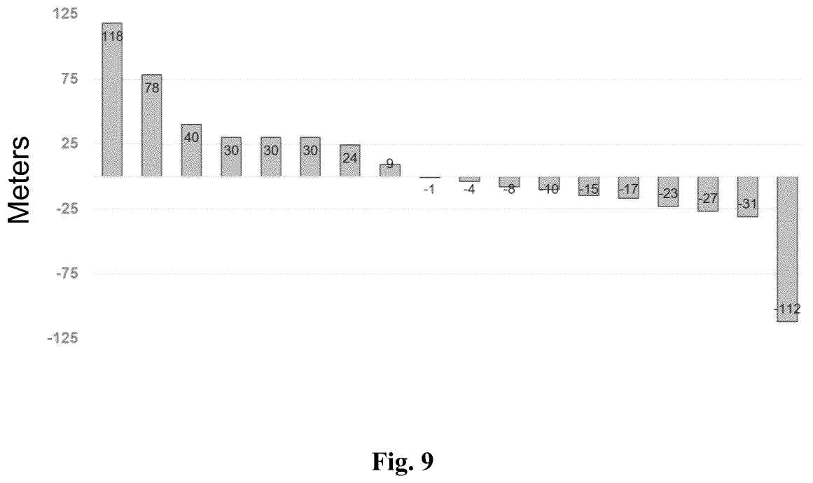

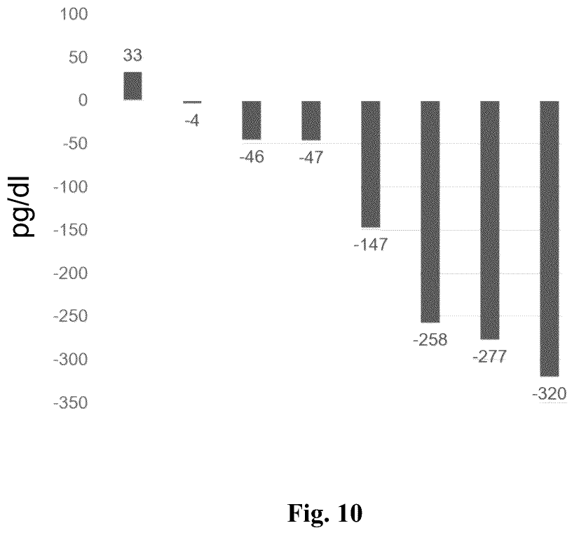

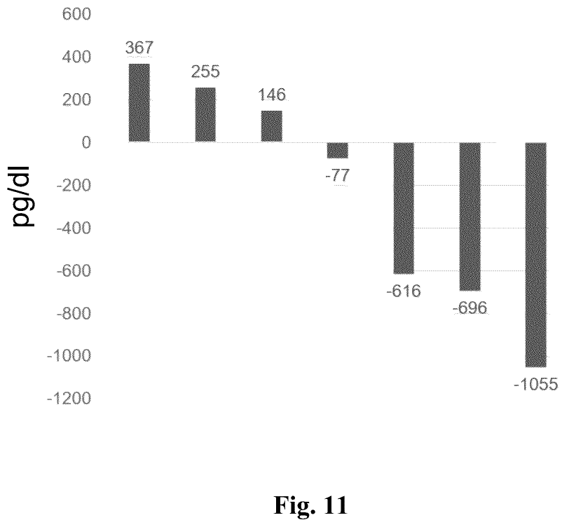

For measures of 6-minute walk distance (6MWD), change in BNP, and change in NT-BNP, see FIGS. 9-11.

### KCCQ Summary Scores

A

| | mean Δ | mean Δ | |
|---|---|---|---|
| Symptom Stability | +9.4 | PHYSICAL LIMITATION | +0.3 |
| Symptom Frequency | +3.1 | SELF EFFICACY | −2.3 |
| Symptom Burden | +6.3 | QUALITY OF LIFE | +4.2 |
| TOTAL SYMPTOM | +4.7 | SOCIAL LIMITATION | +5.5 |
| | | OVERALL SUMMARY | +3.7 |

Summary/Conclusion: The transition to oral levosimendan was well tolerated without safety concerns over a 6-8-week period in patients with PH-HFpEF who had been receiving IV levosimendan for more than 18 months. Oral levosimendan was also associated with further improvements in 6MWD, BNP/NT-ProBNP, and KCCQ scores. Oral levosimendan at 3-4 mg/day appears to be a superior formulation for chronic use in PH-HFpEF.

### REFERENCES

Abraham, William T., et al. (2021) “Effect of empagliflozin on exercise ability and symptoms in heart failure patients with reduced and preserved ejection fraction, with and without type 2 diabetes.” European Heart Journal 42.6: 700-710.
Banfor, P. N., et al. (2008). Comparative effects of levosimendan, OR-1896, OR-1855, dobutamine, and milrinone on vascular resistance, indexes of cardiac function, and 02 consumption in dogs. American Journal of Physiology-Heart and Circulatory Physiology, 294(1), H238-H248.
Bittner, B., et al. (2018). Subcutaneous administration of biotherapeutics: an overview of current challenges and opportunities. BioDrugs, 32(5), 425-440.
Borlaug et al. Levosimendan Improves Hemodynamics And Submaximal Exercise Capacity In PH-HFpEF: Primary Results from the Help-PH-HFpEF Multicenter Randomized Controlled Trial Heart Society of America—Late Breaking Clinical Trials Session, Oct. 3, 2020 4:30 PM-5:30 PM.
Brittain, Evan L., et al. “Elucidating the Clinical Implications and Pathophysiology of Pulmonary Hypertension in Heart Failure With Preserved Ejection Fraction: A Call to Action: A Science Advisory From the American Heart Association.” Circulation 146.7 (2022): e73-e88.
Burkhoff, Daniel, et al. (2021) “Levosimendan improves hemodynamics and exercise tolerance in PH-HFpEF: results of the randomized placebo-controlled HELP trial.” Heart Failure 9.5: 360-370.
Burkhoff et al. “24-hour Levosimendan Infusion Decreases Biventricular Filling Pressures and Increases Cardiac Output at Rest and Exercise in PH-HFpEF.” Circulation 142.Suppl_3 (2020): A15294-A15294.
Chrusciel, P., et al. (2014) Defining the role of trimetazidine in the treatment of cardiovascular disorders: some insights on its role in heart failure and peripheral artery disease. Drugs, 74(9), 971-980.
De Luca, L., et al. (2006). Effects of levosimendan on left ventricular diastolic function after primary angioplasty for acute anterior myocardial infarction: a Doppler echocardiographic study. Journal of the American Society of Echocardiography, 19(2), 172-177.
Dixon, D. D., et al. (2015). Combined post- and pre-capillary pulmonary hypertension in heart failure with preserved ejection fraction. Heart failure reviews, 21(3). 285-297.
Du Toit, E. F., et al. (2008). A role for the RISK pathway and KATP channels in pre- and post-conditioning induced by levosimendan in the isolated guinea pig heart. British journal of pharmacology, 154(1), 41-50.
ElGuindy, A., et al. (2012). Heart failure with preserved ejection fraction. Global Cardiology Science and Practice, 2012(1), 10.
Erdei, N., et al. (2006). The levosimendan metabolite OR-1896 elicits vasodilation by activating the KATP and BKCa channels in rat isolated arterioles. British journal of pharmracology, 148(5), 696-702.
Galie, N., et al. (2009). Guidelines for the diagnosis and treatment of pulmonary hypertension: the Task Force for the Diagnosis and Treatment of Pulmonary Hypertension of the European Society of Cardiology (ESC) and the European Respiratory Society (ERS), endorsed by the International Society of Heart and Lung Transplantation (ISHLT). European heart journal, 30(20), 2493-2537.
Gorter, T. M., et al. (2018). Right heart dysfunction and failure in heart failure with preserved ejection fraction: mechanisms and management. Position statement on behalf of the Heart Failure Association of the European Society of Cardiology. European journal of heart failure, 20(1), 16-37.
Grossini, E., et al. (2005). Hemodynamic effect of intracoronary administration of levosimendan in the anesthetized pig. Journal of cardiovascular pharmacology, 46(3), 333-342.
Haikala, H., et al. (1995). Mechanisms of action of calcium-sensitizing drugs. Journal of cardiovascular pharmacology, 26, S10-9.
Hajjar, R. J., et al. (1991). Calcium-sensitizing inotropic agents in the treatment of heart failure: a critical view. Cardiovascular drugs and therapy, 5(6), 961-965.
Hasenfuss, G., et al. (1998). Influence of the novel inotropic agent levosimendan on isometric tension and calcium cycling in failing human myocardium. Circulation, 98(20), 2141-2147.
Hawthorne, K. M., et al. (2012). Quality assessment in dobutamine stress echocardiography: what are the clinical predictors associated with a non-diagnostic test?. Cardiology research, 3(2), 73.
Hoeper, M. M., et al. (2016). A global view of pulmonary hypertension. The Lancet Respiratory Medicine, 4(4), 306-322.
Jiang, R., et al. (2018). Efficacy and safety of a calcium sensitizer, levosimendan, in patients with right heart failure due to pulmonary hypertension. The clinical respiratory journal, 12(4), 1518-1525.
Kheinen, P., et al. (2001). Levosimendan increases diastolic coronary flow in isolated guinea-pig heart by opening ATP-sensitive potassium channels. Journal of cardiovascular pharmacology, 37(4), 367-374.
Kelly, J. P., et al. (2015) Patient selection in heart failure with preserved ejection fraction clinical trials. Journal of the American College of Cardiology, 65(16), 1668-1682.
Kivikko, M., et al. (2002). Pharmacodynamics and safety of a new calcium sensitizer, levosimendan, and its metabolites during an extended infusion in patients with severe heart failure. The Journal of Clinical Pharmacology, 42(1), 43-51.
Kivikko, M., et al. (2003). Sustained hemodynamic effects of intravenous levosimendan. Circulation, 107(1), 81-86.
Klapholz, M., et al. (2004). Hospitalization for heart failure in the presence of a normal left ventricular ejection fraction: results of the New York Heart Failure Registry. Journal of the American College of Cardiology, 43(8), 1432-1438.
Kleber, F. X., et al. (2009). Repetitive dosing of intravenous levosimendan improves pulmonary hemodynamics in patients with pulmonary hypertension: results of a pilot study. The Journal of Clinical Pharmacology, 49(1), 109-115.
Kostis, J. B., et al. (2004). Omapatrilat and enalapril in patients with hypertension: the Omapatrilat Cardiovascular Treatment vs, Enalapril (OCTAVE) trial. American Journal of hypertension, 17(2), 103-111.
Lai, Y. C., et al. (2019). Insights into the pulmonary vascular complications of heart failure with preserved ejection fraction. The Journal of physiology, 597(4), 1143-1156.
Laurent, S., et al. (2000). Antihypertensive effects of fasidotril, a dual inhibitor of neprilysin and angiotensin-converting enzyme, in rats and humans. Hypertension, 35(5), 1148-1153.
Levine, A. R., et al. (2019). Pulmonary vascular disease in the setting of heart failure with preserved ejection fraction. Trends in cardiovascular medicine, 29(4), 207-217.
Louhelainen, M., et al. (2009). Effects of calcium sensiti zer OR-1986 on cardiovascular mortality and myocardial remodelling in hypertensive Dahl/Rapp rats. Acta phvsiologica Polonica, 12(3), 41.
Louhelainen, M., et al. (2010). Effects of the calcium sensitizer OR-1896, a metabolite of levosimendan, on post-infarct heart failure and cardiac remodelling in diabetic Goto-Kakizaki rats. British journal of pharmacology, 160(1), 142-152.
Maytin, M., et al. (2005). Cardioprotection: a new paradigm in the management of acute heart failure syndromes. The American journal of cardiology, 96(6), 26-31.
McLaughlin, V. V., et al. (2009). ACCF/AHA 2009 expert consensus document on pulmonary hypertension: a report of the American College of Cardiology Foundation Task Force on expert consensus documents and the American Heart Association developed in collaboration with the American College of Chest Physicians; American Thoracic Society, Inc.; and the Pulmonary Hypertension Association. Journal of the American College of Cardiology, 53(17), 1573-1619.
Michaels, A. D., et al. (2005). Effects of intravenous levosimendan on human coronary vasomotor regulation, left ventricular wall stress, and myocardial oxygen uptake. Circulation, 111(12), 1504-1509.
Nassif, Michael E., et al. (2021) “Empagliflozin Effects on Pulmonary Artery Pressure in Patients With Heart Failure: Results From the EMBRACE-HF Trial.” Circulation 143.17: 1673-1686.
Northridge, D. B., et al. (1999). Comparison of the short-term effects of candoxatril, an orally active neutral endopeptidase inhibitor, and frusemide in the treatment of patients with chronic heart failure. American heart journal, 138(6), 1149-1157.
Norton, G. R., et al. (1999). Sustained Antihypertensive Actions of a Dual Angiotensin-Converting Enzyme Neutral Endopeptidase Inhibitor, Sampatrilat, in Black Hypertensive Subjects. American journal of hypertension, 12(6), 563-571.
Oktay, A. A., et al. (2013). The emerging epidemic of heart failure with preserved ejection fraction. Current heart failure reports, 10(4), 401-410.
Oldroyd, S. H., et al. (2019). Pulmonary Hypertension.
Oudiz, R. J., et al. (2007). Pulmonary hypertension associated with left-sided heart disease. Clinics in chest medicine, 28(1), 233-241.
Opitz, Christian F., et al. (2016). Pre-capillary, combined, and post-capillary pulmonary hypertension: a pathophysiological continuum. Journal of the American College of Cardiology 68.4: 368-378.
Pataricza, J., et al. (2000). Comparison of the vasorelaxing effect of cromakalim and the new inodilator, levosimendan, in human isolated portal vein. Journal of pharmacy and pharmacology, 52(2), 213-217.
Peacock, A. J., et al. (2004). Pulmonary Circulation: Diseases and Their Treatment. CRC Press.
Pollesello, P., et al. (1994). Binding of a new Ca2+ sensitizer, levosinendan, to recombinant human cardiac troponin C. A molecular modelling, fluorescence probe, and proton nuclear magnetic resonance study. Journal of Biological Chemistry, 269(46), 28584-28590.
Pollesello, P., et al. (2007). The cardioprotective effects of levosimendan: preclinical and clinical evidence. Journal of cardiovascular pharmacology, 50(3), 257-263.
Segreti, J. A., et al. (2008). Evoked changes in cardiovascular function in rats by infusion of levosimendan, OR-1896 [(R)—N-(4-(4-methyl-6-oxo-1,4,5,6-tetraihydropyridazin-3-yl) phenyl) acetamide], OR-1855 [(R)-6-(4-aminophenyl)-5-methyl-4,5-dihvdropyridazin-3 (2H)-one], dobutarine, and milrinone: comparative effects on peripheral resistance, cardiac output, dP/dt, pulse rate, and blood pressure. Journal of Pharmacology and Experimental Therapeutics, 325(1), 331-340.
Shah, S. J., et al. (2016). Phenotype-specific treatment of heart failure with preserved ejection fraction: a multiorgan roadmap. Circulation, 134(1), 73-90.
Simonneau, G., et al. (2009). Updated clinical classification of pulmonary hypertension. Journal of the American College of Cardiology, 54(1 Supplement), S43-S54.
Sorsa, T., et al. (2004). The contractile apparatus as a target for drugs against heart failure: interaction of levosimendan, a calcium sensitiser, with cardiac troponin c. Molecular and cellular biochemistry, 266(1-2), 87-107.
Szilagyi, S., et al. (2004). The effects of levosimendan and OR-1896 on isolated hearts, myocyte-sized preparations and phosphodiesterase enzymes of the guinea pig. European journal of pharmacology, 486(1), 67-74.
Teerlink, J. R., et al. (2016). RELAX-REPEAT: a multicenter, prospective, randomized, double-blind study evaluating the safety and tolerability of repeat doses of serelaxin in patients with chronic heart failure. Journal of Cardiac Failure, 22(8), S14-S15.
Torres, F., et al. (2019). Efficacy and safety of ralinepag, a novel oral IP agonist, in PAH patients on mono or dual background therapy: results from a phase 2 randomised, parallel group, placebo-controlled trial. European Respiratory Journal, 54(4).
Vandoni, M., et al. (2018). Six minute walk distance and reference values in healthy Italian children: A cross-sectional study. PloS one, 13(10).
Virgadamo, S., et al. (2015). Digoxin: A systematic review in atrial fibrillation, congestive heart failure and post myocardial infarction. World journal of cardiology, 7(11), 808.
Wait, J. C., et al. (2006). Metabolism of [14C] gemopatrilat after oral administration to rats, dogs, and humans, Drug metabolism and disposition, 34(6), 961-970.
Yancy, C. W., et al. (2013). 2013 ACCF/AHA guideline for the management of heart failure: a report of the American College of Cardiology Foundation/American Heart Association Task Force on Practice Guidelines. Journal of the American College of Cardiology, 62(16), e147-e239.
Yancy, C. W., et al. (2013). 2013 ACCF/AHA guideline for the management of heart failure: executive summary: a report of the American College of Cardiology Foundation/American Heart Association Task Force on Practice Guidelines. Circulation, 128(16), 1810-1852.
Yokoshiki, H., et al. (1997). Levosimendan, a novel Ca2+ sensitizer, activates the glibenclamide-sensitive K+ channel in rat arterial myocytes. European journal of pharmacology, 333(2-3), 249-259.

1. A method for treating Pulmonary Hypertension Heart Failure with preserved ejection fraction (PH-HFpEF) in a human subject afflicted with PH-HFpEF comprising orally administering to the human subject an amount of levosimendan, its metabolites OR-1896 or OR-1855, or a combination thereof, that is effective to treat the PH-HFpEF in the human subject, wherein the treating comprises
a reduction in the human subject's serum brain natriuretic peptide (BNP) level and/or N-terminal prohormone of brain natriuretic peptide (NT-proBNP) levels;
an improvement in quality of life as assessed by Kansas City Cardiomyopathy Questionnaire total symptom score (KCCQ-TS), clinical summary score (KCCQ-CS), and/or overall summary score (KCCQ-OS); and/or
a change in resting heart rate of about 5 beats/minute or less and/or a change in systolic arterial blood pressure of about 4 mmHg or less.

2. The method of claim 1, wherein the treating comprises
a) a reduction in the human subject's pulmonary capillary wedge pressure at rest by 1 to 30 mmHg;
b) a stabilization of the human subject's pulmonary capillary wedge pressure at rest at 5 to 35 mmHg or 10 to 35 mmHg;
c) a reduction in the human subject's pulmonary capillary wedge pressure during exercise by the human subject by subject by 1 to 40 mmHg;
d) stabilization of the human subject's pulmonary capillary wedge pressure during exercise by the human subject at 10 to 50 mmHg;
e) the treating does not comprise a significant change in pulmonary capillary wedge pressure during exercise by the human subject;
f) a reduction in the human subject's pulmonary capillary wedge pressure when the human subject's legs are elevated, wherein the reduction is 1 to 30 mmHg;
g) stabilization of the human subject's pulmonary capillary wedge pressure when the human subject's legs are elevated, wherein the stabilization is at 10 to 50 mmHg;
h) a reduction in the human subject's right atrial pressure at rest by 1 to 30 mmHg;
i) stabilization of the human subject's right atrial pressure at rest at 1 to 30 mmHg or at 5 to 30 mmHg;
j) a reduction in the human subject's right atrial pressure during exercise by the human subject by 1 to 30 mmHg;
k) stabilization of the human subject's right atrial pressure during exercise by the human subject at 5 to 40 mmHg;
l) a reduction in the human subject's right atrial pressure when the human subject's legs are elevated;
m) a reduction in the human subject's mean pulmonary artery pressure at rest by 1 to 30 mmHg;
n) stabilization of the human subject's mean pulmonary artery pressure at rest at 15 to 65 mmHg;
o) a reduction in the human subject's mean pulmonary artery pressure during exercise by the human subject by 1 to 30 mmHg;
p) stabilization of the human subject's mean pulmonary artery pressure during exercise by the human subject at 25 to 85 mmHg or 25 to 80 mmHg;
q) a reduction in the human subject's mean pulmonary artery pressure when the human subject's legs are elevated;
r) an increase in the human subject's cardiac output at rest by 0.01 to 3 liters/min;
s) stabilization of the human subject's cardiac output at rest at 2 to 10 liters/min;
t) an increase in the human subject's cardiac output during exercise by the human subject;
u) an increase in the human subject's cardiac output during exercise by the human subject by 0.01 to 5 liters/min or by 0.01 to 4 liters/min or by at 3.0 to 15.0 liters/min;
v) a lack of a significant increase in the human subject's heart rate or does not comprise an increase in the human subject's heart rate of more than 10 beats/min;
w) an improvement in the human subject's quality of life;
x) an improvement in the human subject's six (6) minute walk distance of 5 to 150 meters;
y) an improvement in the physician's assessment of the human subject's functional class;
z) a reduction in the incidence of hospitalization for heart failure;
aa) a reduction in all-cause mortality; or
bb) an improvement in right heart failure and/or right ventricular dysfunction as evidenced by a reduction in right atrial pressure at rest and during 25 watts of exercise.

3. The method of claim 2, wherein in (w) the improvement in the human subject's quality of life is measured by a patient reported outcome assessment tool, wherein the treating comprises an improvement in the human subject's quality of life according to a change in the human subject's patient reported outcome assessment tool score of at least 1, more preferably at least 2.

4. (canceled)

5. The method of claim 1, wherein the human subject is a responder to levosimendan therapy, wherein
a) a responder to levosimendan therapy is a human subject whose pulmonary capillary wedge pressure decreases by at least 4 mmHG during bicycle exercise at 25 watts following the initial administration;
b) a responder to levosimendan therapy is a human subject whose cardiac index decreases by no more than 10% between the baseline measurements and repeated measurements following the initial administration;
c) the human subject is a responder to levosimendan therapy if the human subject has cardiac reserve;
d) the human subject is a responder to levosimendan therapy if the human subject's stroke volume increases during exercise by the human subject;
e) the human subject is a responder to levosimendan therapy if the human subject's stroke volume increases during exercise by the human subject when determined with a catheter in the human subject's heart measuring the blood moving out of the left ventricle with every beat;
f) the human subject is a responder to levosimendan therapy if the human subject's stroke volume increases during exercise by the human subject when estimated with an electrocardiogram and/or echocardiogram;
g) the human subject is a responder to levosimendan therapy if the human subject's stroke volume increases during exercise by the human subject when determined with a dobutamine stress test;
h) the human subject is a responder to levosimendan therapy if the human subject's stroke volume increases during exercise by the human subject by at least 0.005 liters;
i) the human subject is a responder to levosimendan therapy if the human subject's stroke volume increases during exercise by the human subject by 1 to 50 mL when determined with a catheter in the human subject's heart measuring the blood moving out of the left ventricle with every beat;
j) the human subject is a responder to levosimendan therapy if the human subject's stroke volume increases during exercise by the human subject by 1 to 50 mL when estimated with an echocardiogram, right heart catheterization, or other means; or
k) the human subject is a responder to levosimendan therapy if the human subject's stroke volume increases during exercise by the human subject by 1 to 50 mL when determined with a dobutamine stress test; and/or
wherein the human subject afflicted with PH-HFpEF
a) has a left ventricular ejection fraction of at least 40%;
b) has a baseline pulmonary arterial pressure of at least 35;
c) has a baseline pulmonary capillary wedge pressure of at least 20;
d) is classified as classification IIb or classification III by the physician's assessment of New York Heart Association Classification;
e) has the ability to walk at least 50 meters in a six-minute walk test, does not have the ability to walk more than 550 meters in a six-minute walk test, or has the ability to walk at least 50 meters, but not more than 550 meters, in a six-minute walk test;
f) is not afflicted with heart failure with reduced ejection fraction;
g) is not afflicted with heart failure with preserved ejection fraction without pulmonary hypertension;
h) has a primary diagnosis of Group 2 PH-HFpEF;
i) is not afflicted with coronary artery disease;
j) has not had previous percutaneous coronary intervention;
k) has not had previous percutaneous coronary intervention, unless the human subject has had a negative stress test within the last year;
l) has not had previous cardiac surgery;
m) has not had previous cardiac surgery, unless the human subject has had a negative stress test within the last year;
n) is not afflicted with congenital heart disease;
o) is not afflicted with a clinically significant lung disease;
p) does not have a planned heart or lung surgery;
q) does not have a cardiac index greater than 4.0 L/min/m2;
r) does not concomitantly receive pulmonary vasodilator therapy;
s) has not received pulmonary vasodilator therapy within the last 14 days;
t) does not receive dialysis treatment;
u) does not have a Glomerular Filtration Rate less than 30 mL/min/1.73 m2;
v) does not have liver dysfunction with Child Pugh Class B or C;
w) does not have evidence of systemic infection;
x) does not weigh more than 150 kg;
y) can manage their symptomatic systolic blood pressure to ensure it is greater than 100 mmHg;
z) does not have a heart rate greater than or equal to 100 beats per minute with the drug;
aa) does not have a heart rate greater than or equal to 100 beats per minute with the drug that is symptomatic and persistent for at least 10 minutes;
bb) does not have hemoglobin less than 80 g/L;
cc) does not have serum potassium less than 3.0 mmol/L at baseline;
dd) does not have serum potassium greater than 5.5 mmol/L at baseline;
ee) does not have serum potassium less than 3.0 mmol or greater than 5.5 mmol/L at baseline;
ff) does not have severely compromised immune function;
gg) is not pregnant, is not suspected to be pregnant, or is not breast-feeding; or
hh) is a patient with Biventricular Failure; and/or
wherein the administering takes place once daily, twice daily, three times daily, four times daily, intermittently, weekly, or chronically; and/or
wherein the oral administration comprises an immediate release formulation, modified release formulation, or an extended-release formulation; and/or
wherein the amount of levosimendan its metabolites OR-1896 or OR-1855, or a combination thereof, is administered in combination with a cardiovascular drug.

6-10. (canceled)

11. The method of claim 5, wherein the amount of levosimendan its metabolites OR-1896 or OR-1855, or a combination thereof, and the amount of the cardiovascular drug when taken together is effective to reduce the symptoms of PH-HFpEF;
wherein the cardiovascular drug is a drug used to treat pulmonary arterial hypertension (PAH), World Health Organization (WHO) Groups 1-5 pulmonary hypertension patients, coronary artery disease (CAD), or heart failure with reduced ejection fraction (HFrEF);
wherein the cardiovascular drug is a PDE inhibitor, a phosphodiesterase-5 (PDE5) inhibitor, an endothelin receptor antagonist (ERA), a prostanoid, a soluble guanylate cyclase stimulator, a nitrate, a nitrite, an NO donor, a calcium channel blocker (CCB), a fatty acid oxidation inhibitor, a beta-blocker (BB), an angiotensin-converting enzyme (ACE) inhibitor, a neprilysin inhibitor, a neprilysin and angiotensin receptor blocker (ANRI), an angiotensin II receptor blocker (ARB), a diuretic, an aldosterone antagonist, digoxin, ivabradine, hydralazine, seralaxin, a natriuretic peptide, an atrial natriueretic peptide (ANP), a natriuretic peptide, a K-ATP channel activator, a NEP inhibitor, or a prostacyclin; and/or
wherein the cardiovascular drug is a pulmonary vasodilator drug; wherein the pulmonary vasodilator is a phosphodiesterase-5 (PDE5) inhibitor, an endothelin receptor antagonist (ERA), or a prostacyclin; and/or wherein the amount of levosimendan, its metabolites OR-1896 or OR-1855, or a combination thereof, administered in combination with the pulmonary vasodilator drug is administered to a human subject afflicted with pre and post capillary pulmonary hypertension and heart failure with preserved ejection fraction (Cpc-PH-HFpEF).

12-16. (canceled)

17. The method of claim 1, wherein no atrial rest or ventricular rest is observed when comparing baseline electrocardiographic monitoring with 72-hour monitoring after 5 weeks of treatment;
wherein treating presents no more statistically significant adverse events than the matching placebo; and/or
wherein the subject is orally administered a capsule comprising up to 0.1 mg, 0.25 mg, 0.5 mg, 0.75 mg, 1 mg, 2 mg, 3 mg, or 4 mg of levosimendan, its metabolites OR-1896 or OR-1855, or a combination thereof.

18-19. (canceled)

20. The method of claim 17, wherein the subject is administered a capsule once a day, twice a day, three times a day, or four times a day for a time period of 1-60 days;
wherein the subject increases the number of capsules taken per day after every time period if the treatment is tolerated by the subject;
wherein the subject is orally administered between 0.1-10 mg of levosimendan, its metabolites OR-1896 or OR-1855, or a combination thereof, per day of levosimendan, its metabolites OR-1896 or OR-1855, or a combination thereof per day; and/or
wherein the subject received a final intravenous injection of levosimendan, its metabolites OR-1896 or OR-1855, or a combination thereof at least one day before beginning oral administration of levosimendan, its metabolites OR-1896 or OR-1855, or a combination thereof.

21-23. (canceled)

24. The method of claim 1, wherein the human subject is administered an effective amount of a combination therapy comprising
a) an amount of levosimendan, its metabolites OR-1896 or OR-1855, or a combination thereof; and
b) an amount of a sodium-glucose cotransporter-2 (SGLT-2) inhibitor.

25. The method of claim 24, comprising periodically administering to the subject an amount of the levosimendan, its metabolites OR-1896 or OR-1855, or a combination thereof, and an amount of the SGLT-2 inhibitor, wherein the amounts when taken together are effective to treat the subject;
wherein treating the subject with the combination therapy is more effective to treat the subject than when either the amount of levosimendan or the amount of the SGLT-2 inhibitor is administered alone; and/or
wherein the amounts of levosimendan and the SGLT-2 inhibitor when taken together are effective to achieve a greater than additive therapeutic result in treating the subject.

26-27. (canceled)

28. The method of claim 24, wherein the subject was receiving a therapy including levosimendan, its metabolites OR-1896 or OR-1855, or a combination thereof, prior to initiating a SGLT-2 inhibitor therapy; or
wherein the subject was receiving a SGLT-2 inhibitor therapy prior to initiating a therapy including levosimendan, its metabolites OR-1896 or OR-1855, or a combination thereof.

29. (canceled)

30. The method of claim 24, wherein the amount of SGLT-2 inhibitor is administered first, followed by administration of the amount of levosimendan, its metabolites OR-1896 or OR-1855, or a combination thereof; or
wherein the amount of levosimendan, its metabolites OR-1896 or OR-1855, or a combination thereof, is administered first, followed by administration of a SGLT-2 inhibitor.

31. (canceled)

32. The method of claim 24, wherein the levosimendan, its metabolites OR-1896 or OR-1855, or a combination thereof, and the SGLT-2 inhibitor are administered sequentially or simultaneously;
wherein the levosimendan, its metabolites OR-1896 or OR-1855, or a combination thereof, and the SLGT-2 inhibitor are administered periodically, chronically, weekly, or intermittently;
wherein the SGLT-2 inhibitor is administered orally; and/or
wherein the SGLT-2 inhibitor is selected from the group consisting of empagliflozin, canagliflozin, ertugliflozin, or dapagliflozin;
wherein the subject is administered between 10-25 mg empagliflozin per day;
wherein the subject is administered between 5-10 mg dapagliflozin per day;
wherein the subject is administered between 100-300 mg canagliflozin per day; or
wherein the subject is administered between 5-15 mg ertugliflozin per day.

33-40. (canceled)

41. The method of claim 24, wherein the subject is administered between 0.1-10 mg of levosimendan, its metabolites OR-1896 or OR-1855, or a combination thereof, per day of levosimendan, its metabolites OR-1896 or OR-1855, or a combination thereof per day; and/or
wherein the combination therapy is administered as a fixed dose combination.

42. (canceled)

43. The method of claim 24, wherein the treating with the combination therapy comprises providing
a) an improvement in the human subject's quality of life;
b) an improvement in the human subject's exercise capacity;
c) an improvement in a physician's assessment of the human subject's functional class;
d) a reduction in the incidence of hospitalization for heart failure; and/or
e) a reduction in cardiovascular death; and/or
wherein the treating comprises providing an improvement in the human subject's hemodynamic measurements at rest and exercise.

44. (canceled)

45. The method of claim 43, wherein the improvement in the subject's exercise capacity is an increase of at least 10, 20, 30, 40, 50, 60, 70, 80, or 100 meters in a 6-minute walk distance compared to a baseline 6-minute walk distance before the combination therapy treatment;
wherein the improvement in the subject's exercise capacity is an increase of at least 10%, 20%, 30%, 40%, or 50% relative to a baseline 6-minute walk distance before the combination therapy treatment; and/or
wherein the improvement in the subject's exercise capacity is within one, two, three, four, five, six, seven, eight, nine, ten, twenty, thirty, forty, or fifty weeks of the administration of the combination therapy.

46-48. (canceled)

49. The method of claim 1, wherein the subject is transitioned to oral administration of levosimendan from intravenous administration of levosimendan, and the OR-1896 plasma concentration of the subject remains the same or increases after the transition to oral administration of levosimendan.

50. A pharmaceutical composition comprising levosimendan, its metabolites OR-1896 or OR-1855, or a combination thereof, a SGLT-2 inhibitor, and a pharmaceutically acceptable carrier, wherein the SGLT-2 inhibitor is selected from the group consisting of empagliflozin, canagliflozin, ertugliflozin, and dapagliflozin;
a pharmaceutical composition comprising an amount of a SGLT-2 inhibitor and an amount of levosimendan, its metabolites OR-1896 or OR-1855, or a combination thereof, for use in treating a subject afflicted with PH-HFpEF, wherein the SGLT-2 inhibitor and the levosimendan, its metabolites OR-1896 or OR-1855, or a combination thereof, are administered simultaneously, contemporaneously or concomitantly, wherein the SGLT-2 inhibitor is selected from the group consisting of empagliflozin, canagliflozin, ertugliflozin, and dapagliflozin;
a pharmaceutical composition in unit dosage form, useful in treating a subject afflicted with PH-HFpEF, which comprises:
a) an amount of levosimendan; and
b) an amount of an SGLT-2 inhibitor,
wherein the respective amounts of said levosimendan and said SGLT-2 inhibitor in said composition are effective, upon concomitant administration to said subject of one or more said unit dosage forms of said composition, to achieve a greater than additive therapeutic result in treating the subject, wherein the SGLT-2 inhibitor is selected from the group consisting of empagliflozin, canagliflozin, ertugliflozin, and dapagliflozin; a package comprising:
a. a first pharmaceutical composition comprising an amount of levosimendan and a pharmaceutically acceptable carrier;
b. a second pharmaceutical composition comprising an amount of an SGLT-2 inhibitor and a pharmaceutically acceptable carrier; and
c. instructions for use of the first and second pharmaceutical compositions together to treat a subject afflicted with PH-HFpEF,
wherein the SGLT-2 inhibitor is selected from the group consisting of empagliflozin, canagliflozin, ertugliflozin, and dapagliflozin;
a therapeutic package for dispensing to, or for use in dispensing to, a subject afflicted with PH-HFpEF, which comprises:
one or more unit doses, each such unit dose consisting essentially of:
i) an amount of levosimendan; and
ii) an amount of an SGLT-2 inhibitor,
wherein the respective amounts of said levosimendan and said SGLT-2 inhibitor in said unit dose are effective, upon concomitant administration to said subject, to achieve a greater than additive therapeutic result in treating the subject, and a finished pharmaceutical container therefor, said container containing said unit dose or unit doses, said container further containing or comprising labeling directing the use of said package in the treatment of said subject,
wherein the SGLT-2 inhibitor is selected from the group consisting of empagliflozin, canagliflozin, ertugliflozin, and dapagliflozin;
a medicament comprising an amount of an oral formulation of levosimendan, its metabolites OR-1896 or OR-1855, or a combination thereof, for use in effectively treating Pulmonary Hypertension Heart Failure with preserved ejection fraction (PH-HFpEF) in a human subject; or
a medicament comprising an amount of an oral formulation of levosimendan, its metabolites OR-1896 or OR-1855, or a combination thereof, for use in combination with a cardiovascular drug to effectively treat Pulmonary Hypertension Heart Failure with preserved ejection fraction (PH-HFpEF) in a human subject.

51. (canceled)

52. Use of a SGLT-2 inhibitor in combination or as an add-on with a therapy that includes levosimendan, its metabolites OR-1896 or OR-1855, or a combination thereof, to treat a subject afflicted with PH-HFpEF, wherein the SGLT-2 inhibitor and the levosimendan, its metabolites OR-1896 or OR-1855, or a combination thereof are administered simultaneously, contemporaneously or concomitantly, wherein the SGLT-2 inhibitor is selected from the group consisting of empagliflozin, canagliflozin, ertugliflozin, and dapagliflozin;
use of a SGLT-2 inhibitor in the manufacturing of a medicament for use in combination with or as an add-on to a therapy that includes levosimendan, its metabolites OR-1896 or OR-1855, or a combination thereof, to treat a subject afflicted with PH-HFpEF, wherein the SGLT-2 inhibitor and the levosimendan, its metabolites OR-1896 or OR-1855, or a combination thereof are administered simultaneously, contemporaneously or concomitantly, wherein the SGLT-2 inhibitor is selected from the group consisting of empagliflozin, canagliflozin, ertugliflozin, and dapagliflozin; or
use of an amount of an oral formulation of levosimendan, its metabolites OR-1896 or OR-1855, or a combination thereof, to effectively treat Pulmonary Hypertension Heart Failure with preserved ejection fraction (PH-HFpEF) in a human subject;
use of an amount of an oral formulation of levosimendan, its metabolites OR-1896 or OR-1855, or a combination thereof, for preparing medicament for administering to a human subject afflicted with Pulmonary Hypertension Heart Failure with preserved ejection fraction (PH-HFpEF to effectively treat PH-HFpEF in the human subject;
use of an amount of an oral formulation of levosimendan, its metabolites OR-1896 or OR-1855, or a combination thereof, in combination with a cardiovascular drug to effectively treat Pulmonary Hypertension Heart Failure with preserved ejection fraction (PH-HFpEF) in a human subject; or
use of an amount of an oral formulation of levosimendan, its metabolites OR-1896 or OR-1855, or a combination thereof, for preparing a medicament in combination with a cardiovascular drug for administering to a human subject afflicted with Pulmonary Hypertension Heart Failure with preserved ejection fraction (PH-HFpEF) to effectively treat PH-HFpEF in the human subject.

53-69. (canceled)

70. The method of claim 1, wherein the subject is chronically administered 3-4 mg of levosimendan, its metabolites OR-1896 or OR-1855, or a combination thereof, per day; the levosimendan, its metabolites OR-1896 or OR-1855, or a combination thereof is orally administered in the form of a pill, capsule, tablet, or liquid; and/or wherein the levosimendan, its metabolites OR-1896 or OR-1855, or a combination thereof is administered at a dose of 1 mg TID or QID.

71. The method of claim 1, wherein the KCCQ-TS score is improved by 4-5 points, the KCCQ-CS score is improved by up to 2-3 points, and/or KCCQ-OS score is improved by up to 3-4 points; the human subject's BNP level is reduced by up to 100-150 pg/dl; and/or the human subject's NT-proBNP level is decreased 200-250 pg/dl.
